(19) United States
(12) Patent Application Publication
(10) Pub. No.: US 2021/0311032 A1
(43) Pub. Date: Oct. 7, 2021

(54) SYSTEMS AND METHODS FOR ALLERGEN DETECTION

(71) Applicant: DOTS Technology Corp., Natick, MA (US)

(72) Inventors: Adi Gilboa-Geffen, Wayland, MA (US); Alan Lloyd Weeks, S. Easton, MA (US); Valerie Villareal, Boston, MA (US); Patrick Murphy, Allston, MA (US); Eric Anthony Robertson, San Antonio, TX (US); David Carpenter, Jaffrey, NH (US); Deirdre Ellen Day, Winchester, MA (US); Matthew Bernard Dean, Far Hills, NJ (US); Todd Glendon Campbell, Holliston, MA (US); Gregory J. Kintz, Santa Cruz, CA (US); Paul Koh, New York, NY (US); David Jennings Dostal, Hanover, NH (US); Kevin Doherty, San Francisco, CA (US); Joel F. Jensen, Redwood City, CA (US); William Law, Palo Alto, CA (US); Russell C. Mead, Jr., Chapel Hill, NC (US); J. Efraín Alcorta, Austin, TX (US)

(21) Appl. No.: 16/971,701

(22) PCT Filed: Feb. 21, 2019
(86) PCT No.: PCT/US2019/018860

§ 371 Date: Aug. 21, 2020

Related U.S. Application Data
(60) Provisional application No. 62/633,126, filed on Feb. 21, 2018.
(60) Provisional application No. 62/687,126, filed on Jun. 19, 2018.

(51) Int. Cl. G01N 33/53; G01N 33/543; C12Q 1/6816; G01N 33/02

(52) U.S. Cl. CPC G01N 33/5308; G01N 33/02; C12Q 1/6816; G01N 33/54366

(57) ABSTRACT

The present invention is drawn to devices and systems for allergen detection in food samples. The allergen detection system includes a disposable analysis cartridge and a detection device with an optimized optical system.

### FIELD OF THE INVENTION

The present invention is drawn to portable devices and systems for allergen detection in samples such as food samples. The invention also provides methods for detecting the presence and/or absence of an allergen in a sample.

### BACKGROUND OF THE INVENTION

Allergy (e.g., food allergy) is a common medical condition. It has been estimated that in the United States, up to 2 percent of adults and up to 8 percent of children, particularly those under three years of age, suffer from food allergies (about 15 million people), and this prevalence is believed to be increasing. A portable device that enables a person who has a food allergy to test their food and determine accurately and immediately the allergen content will be of great benefit to provide for an informed decision on whether to consume or not.

Researchers have tried to develop suitable devices and methods to meet this need, such as those devices and systems disclosed in U.S. Pat. No. 5,824,554 to McKay; US Patent Application Pub. No.: 2008/0182339 and U.S. Pat. No. 8,617,903 to Jung et al.; US Patent Application Pub. No.: 2010/0210033 to Scott et al; U.S. Pat. No. 7,527,765 to Royds; U.S. Pat. No. 9,201,068 to Suni et al.; and U.S. Pat. No. 9,034,168 to Khattak and Sever. There is still a need for improved molecule detection technologies. There is also a need for devices and systems that detect allergens of interest in less time, with high sensitivity and specificity, and with less technical expertise than the devices used today.

The present invention provides a portable detection device for fast and accurate detection of an allergen in a sample by using aptamer-based signal polynucleotides (SPNs). The SPNs, as detection agents, specifically bind to the allergen of interest, forming SPN: protein complexes. The sensor to capture the SPNs may comprise a chip printed with nucleic acid molecules that hybridize to the SPNs (e.g., DNA chip). The detection system may comprise a separate sampler, disposable cartridges/vessels for processing the sample and implementing the detection assay, and a detection instrument including an optical system for operating the detection and detecting the reaction signal. The detection agents (e.g., SPNs) and sensors (e.g., DNA chips) may be integrated into the disposable cartridges of the present invention. The cartridges, detection agents and the detection sensors may also be used in other detection systems. Other capture agents such as antibodies specific to allergen proteins may also be used in the present detection systems. Such devices may be used by consumers in non-clinical settings, for example in the home, in restaurants and any other facility serving food.

### SUMMARY OF THE INVENTION

The present invention provides systems, devices, disposable vessels/cartridges, optical systems and methods for use in molecule detection in various types of samples, particularly allergens in food samples. The allergen detection devices and systems are portable and handheld.

One aspect of the present invention is an assembly for detecting a molecule of interest in a sample. The assembly comprises a sample processing cartridge configured to accept the sample for processing to a state permitting the molecule of interest to engage in an interaction with a detection agent. The assembly includes a detector unit configured to accept the sample processing cartridge in a configuration which permits a detection mechanism housed by the detector unit to detect the interaction of the molecule of interest with the detection agent. The interaction triggers a visual indication on the detector unit that the molecule of interest is present or absent in the sample.

The molecule of interest may be a protein or functional fragment thereof, a nucleic acid molecule, or a polysaccharide or functional fragment thereof. In some embodiments, the molecule of interest may be an allergen such as a food allergen. Allergens are antigens (portions or functional fragments of molecules such as proteins and polysaccharides) which elicit an immune response resulting in an allergy condition.

In some embodiments, the detection agent is an antibody or variant thereof, a nucleic acid molecule or a small molecule. In some embodiments where the detection agent is a nucleic acid molecule comprising a nucleic acid sequence that binds to the molecule of interest. The nucleic acid-based detection agent may be a signaling polynucleotide derived from an aptamer comprising a nucleic acid sequence that binds to the molecule to be detected.

In some embodiments, the sample processing cartridge comprises a homogenizer configured to produce a homogenized sample, thereby releasing the molecule of interest from a matrix of the sample into an extraction buffer in the presence of the detection agent. The sample processing cartridge also comprises a first conduit to transfer the homogenized sample and detection agent through a filter system to provide a filtrate containing the molecule of interest and the detection agent and a second conduit to transfer the filtrate to a detection chamber with a window. The detection mechanism of the detector unit analyzes the detection chamber through the window to identify the interaction of the molecule of interest with the detection agent in the detection chamber.

The homogenizer may be powered by a motor located in the detector unit with the motor functionally coupled to the homogenizer when the sample processing cartridge is accepted by the detector unit.

The sample processing cartridge may further include a chamber holding wash buffer for washing the detection chamber and a waste chamber for accepting outflow contents of the detection chamber after washing.

In some embodiments, the sample processing cartridge further comprises a rotary valve switching system for providing a plurality of fluid flow paths and channels for transfer of the homogenized sample to the filter system, for transfer of the filtrate to the detection chamber, for transfer of the wash buffer to the detection chamber and for transfer of contents of the detection chamber to the waste chamber. The rotary valve switching system may be further configured to provide a closed position to prevent fluid movement in the sample processing cartridge. In some embodiments, the rotary valve switching system may be powered by a motor located in the detector unit with the motor functionally coupled to the rotary valve system when the sample processing cartridge is accepted by the detector unit.

In some embodiments, the detection chamber includes a transparent substrate with a detection probe molecule immobilized thereon. The detection probe is configured to engage in a probe interaction with the detection agent. An interaction of the molecule of interest with the detection agent prevents the detection agent from engaging in the probe interaction with the detection probe. The transparent substrate may further include an optically detectable control probe molecule immobilized thereon, for normalization of signal output measured by the detection mechanism. In some embodiments, the transparent substrate includes two different optically detectable control probe molecules immobilized thereon, for normalization of signal output measured by the detection mechanism. The control probe molecule is a nucleic acid molecule that does not bind to the molecule of interest, nor the detection agent. In some embodiments, the substrate may be a glass chip.

In some embodiments, the detection agent includes an optically-detectable moiety which is activated when the probe interaction is engaged. The optically-detectable moiety may be a fluorescent moiety.

In some embodiments, the detection mechanism housed by the detector unit is a fluorescence detection system with a laser for excitation of fluorescence, the fluorescence detection system configured for detection of a fluorescence emission signal and/or a fluorescence scatter signal when the probe interaction is engaged and subjected to laser excitation. The detection mechanism may include a plurality of optical elements placed within a stepped bore in the detector unit in either a straight or a folded arrangement.

In some embodiments, the detector unit further comprises a signal processor for analyzing fluorescence emission signal and/or a fluorescence scatter signal to identify the probe interaction and transmit the identity of the molecule of interest, or a source of the molecule of interest to the visual indication such that an operator of the assembly is informed of the presence or absence of the molecule of interest or a source of the molecule of interest in the sample.

In some embodiments, the transparent substrate comprises a plurality of different detection probe molecules for detection of a plurality of different detection agents configured to provide a plurality of different interactions with different molecules of interest.

In some embodiments, the sample processing cartridge further comprises a sample concentrator for concentrating the filtrate prior to transfer of the filtrate to the detection chamber.

In some embodiments, the assembly further includes a sampler. The sampler includes a hollow tube with a cutting edge for cutting a source to generate and retain the sample as a core within the hollow tube. This embodiment of the sampler also has a plunger for pushing the sample out of the hollow tube and into a port in the sample processing cartridge.

Another aspect of the present invention is directed to a detection system and device for detecting the presence and/or absence of one or more allergens of interest in a sample. In various embodiments, the detection system comprises at least one disposable processing cartridge configured to accept a test sample and to process the sample to a state that permits the allergen of interest in the sample to engage in the interaction with a detection agent, and an integrated detector unit configured to accept the disposable cartridge and to operate the sample process for detection of the interaction between the allergen of interest and the detection agent inside the disposable cartridge. The detector unit may be removably connected to the disposable cartridge. In some embodiments, the system may further comprise a sampler for collecting a test sample and transferring the collected sample to the sample processing cartridge.

In some embodiments, the sampler for collecting a test sample is a food corer including a hollow tube with a cutting edge for cutting a source to generate and retain the sample as a core within the hollow tube and a plunger for pushing the sample out of the hollow tube and into a port in the sample processing cartridge. The corer may be operatively connected to the disposable cartridge for transferring a collected test sample to the cartridge.

In some embodiments, the disposable processing cartridge may comprise (i) a sample receiving chamber with a homogenizer configured to homogenize the sample with an extraction buffer in the presence of the detection agent, thereby permitting the allergen of the interest in the sample to engage in the interaction with the detection agent, (ii) a filter system configured to provide a filtrate containing the allergen of interest and the detection agent, (iii) a detection chamber with a window, wherein the detection chamber includes a separate substrate with a detection probe molecule immobilized thereon, (iv) a chamber holding wash buffer for washing the detection chamber, (v) a waste chamber for accepting and storing outflow contents of the detection chamber after washing, (vi) a rotary valve switching system and conduits configured to transfer the homogenized sample and detection agent through the filter system, to transfer the filtrate to the detection chamber, and to transfer the wash buffer to the detection chamber and outflow contents from the detection chamber to the waste chamber, and (vii) an air flow system configured to regulate air pressure and flow rate in the cartridge.

In some examples, the disposable processing cartridge is configured to detect one particular allergen. In other examples, the sample processing cartridge is configured to detect more than one allergen.

In some embodiments, the detector unit may comprise an outer housing with a receptacle to the disposable processing cartridge and an execution button to execute the process. The detector unit is thereby configured to drive the detection process. In some embodiments, the detector unit may comprise (i) a motor configured to drive the homogenizer of the cartridge, (ii) a motor configured to drive the rotary valve switching system of the cartridge, (iii) a pump configured to driving the flow of fluids in the cartridge, (iv) a detection mechanism to detect the interaction of the allergen of the interest and the detection agent wherein the interaction triggers a visual indication on a display of the detector unit that the allergen of interest is present or absent and (v) a display window to allow the operator to view the detection result.

In some embodiments, the filter system of the sample processing cartridge is a filter assembly comprising a bulk filter and a membrane filter. The bulk filter may comprise a gross filter and a depth filter with a cotton volume to filtrate gross debris from the processed sample. The membrane has a pore size from 1 μm to 2 μm. In some embodiments, the filter assembly may further comprise a filter cap that can lock the rotary valve.

In some embodiments, the sample processing cartridge comprises the detection agent that can specifically bind to an allergen of the interest. In some embodiments, the detection agent is prestored in the extraction buffer. The detection agent is a nucleic acid molecule comprising a nucleic acid sequence that binds to the allergen of interest, and a fluorescent moiety attached to one end of the nucleic acid sequence. The nucleic acid-based detection agent may be stored in the buffer comprising MgCl₂. In some examples, the detection agent is a signaling polynucleotide (SPN) derived from an aptamer that binds to the target allergen specifically and with high affinity.

In some embodiments, the detection chamber in the cartridge comprises a separate substrate with a detection probe molecule immobilized thereon. The detection probe molecule is configured to engage in the interaction with the detection agent, wherein the interaction of the allergen of interest with the detection agent prevents the detection agent from engaging in the interaction with the probe molecule. In some embodiments, the detection probe is a nucleic acid molecule comprising a short nucleic acid sequence that is complementary to the nucleic acid sequence of the detection agent. In some embodiments where the detection probe molecule is immobilized on a specialized local area of the substrate which is referred to as a reaction panel.

In some embodiments, the substrate further comprises an optically detectable control probe molecule immobilized thereon, for normalization of signal output measured by the detection mechanism. In some examples, the control probe is immobilized on a specialized local area of the substrate which is referred to as a control panel. In some embodiments, the substrate comprises at least one reaction panel and at least two control panels. In one preferred embodiment, the substrate is a glass chip. The detection chamber may comprise at least one optical window that is aligned with the substrate. In one embodiment, the optical window is configured for measuring signal outputs from the interaction of the detection probe with the detection agent by the detection mechanism of the detector unit. In other embodiments, the detection chamber may comprise a separate window configured for measuring scattered light from the substrate by the detection mechanism.

In some embodiments, the disposable processing cartridge may comprise a data chip configured for providing the cartridge information.

In some embodiments, the detection mechanism is a fluorescence detection system configured for detection of a fluorescence emission signal and/or a fluorescence scatter signal from the detection chamber. In some embodiments, the fluorescence detection system comprises (i) a laser for excitation of fluorescence, (ii) a plurality of optical components to guide the laser excitation to the substrate within the detection chamber, (iii) a plurality of collection lens to collect the fluorescence emitted from the substrate, (iv) a fluorescence detector for measuring the emitted light from the substrate, and (v) a signal processor for analyzing fluorescence emission signal and/or fluorescence scatter signal to identify the probe interaction and transmit the identity of the allergen of interest to the visual indication such that an operator is informed of the presence or absence of the allergen of interest in the sample.

In some embodiments, the optical elements of the fluorescence detection system are placed within a stepped bore in the detector unit in either a straight or a folded arrangement.

In some embodiments, a printed circuit board (PCB) may be connected directly or indirectly to the fluorescence detection system for displaying the testing readout. The result may be displayed as numbers, icons, colors and/or letters, or other equivalents.

In one aspect of the invention, the sample processing cartridge is configured to be a disposable test cup or cup-like container. The disposable test cup or cup-like container may be constructed as an analytical module in which a sample is processed and an allergen of interest in the test sample is detected through the interaction with a detection agent. In some embodiments, the disposable test cup or cup-like container comprises (i) a top cover configured to accept the sample and to seal the cup or cup-like container wherein the top cover includes a port for accepting the sample and at least one breather filter that allows air in, and (ii) a body part configured to process the sample to a state permitting the allergen of interest to engage in an interaction with the detection agent and (iii) a bottom cover configured to connect to the cup body part thereby forming a detection chamber with a window at the bottom of the assembled test cup, and to provide the connecting surface to a detector unit. The exterior of the bottom cover comprises a plurality of ports for connecting a plurality of motors located in the detection unit to operate the homogenizer, the rotary valve system and the flow of the fluids. The window of the detection chamber is connected to the detection mechanism in the detector unit.

In some embodiments, the detection chamber in the interior of the bottom cover includes (i) a separate substrate comprising a optically detectable detection probe molecule immobilized thereon that engages in the interaction of the detection agent, (ii) a plurality of fluid paths and (iii) a window wherein a detection mechanism of the detector unit analyzes the interaction between the homogenized sample and the detection probe molecule and identifies the allergen of interest in the sample.

In some embodiments, the cup body part may be divided into several compartments (e.g., chambers) specialized for various functions, including sample collection and homogenization, buffer and reagent storage, filtrate collection, wash, and waste collection. In one embodiment, the cup body part may comprise (i) a chamber with a homogenizer for homogenizing the sample in an extraction buffer, thereby releasing the molecule of interest from a matrix of the sample into the extraction buffer and engaging in the interaction with a detection agent present in the extraction buffer. (ii) a conduit for transferring the homogenized sample through a filter system that is included in the body part to provide a filtrate containing the molecule of interest and the detection agent, (iii) a separate chamber for holding wash buffer for washing the molecule of interest and the detection agent, (iv) a separate chamber for receiving and storing the outcome consents from washing the molecule of interest and the detection agent, (v) a conduit for transferring the filtrate to a detection chamber, and (vi) a rotary valve switching system, fluid paths and vents necessary for fluid flow within the compartments inside the cartridge.

In one aspect of the invention, a fluorescence detection system for detecting a fluorescence signal comprises (i) a laser source configured to provide light excitation energy: (ii) a plurality of optical components configured to guide the laser excitation source to a reaction area of a substrate to form a spot covering said reaction area wherein a detectable probe molecule is immobilized thereon, and to a control area of the same substrate wherein a control probe is immobilized thereon, thereby exciting the detection probe molecule and the control probe immobilized thereon; (iii) a plurality of light collection components configured to collect light energy emitted from the reaction area and the control area of the substrate, respectively; (iv) a fluorescence detector for measuring the emitted light from the reaction area of the substrate and/or from the control areas of the substrate, and (v) a processor for processing the measurements from the fluorescence detector.

Another aspect of the present invention relates to a system for detecting the presence and/or absence of an allergen in a sample, the system comprising: (a) a detector unit comprising an optical system configured to measure fluorescence signal outputs, thereby detecting the presence or absence of the allergen; and (b) a disposable cartridge configured to process the sample, which docks into a receptacle of the detector unit, the cartridge comprising: (1) an upper module comprising a plurality of chambers isolated from each other with each chamber of the plurality of chambers comprising a lower port to permit entry and/or exit of fluids, the plurality of chambers comprising: (i) a homogenization chamber including a rotor for homogenizing the sample and extracting allergens; (ii) a wash buffer chamber; (iii) a waste chamber configured to receive liquid waste; and (iv) a reaction chamber in optical communication with the optical system, for detecting the allergen; and (2) a base configured to connect to the upper module, the base comprising: (i) a plurality of fluid paths joining the lower port of each chamber when the cartridge is inserted into the receptacle; and (ii) a valve configured to form a plurality of bridging fluid connections between individual fluid paths of the plurality of fluid paths, thereby allowing selective fluid movement into and/or out of the plurality of chambers.

In some embodiments of the system, the plurality of bridging fluid connections comprises: (a) a first fluid connection between the wash buffer chamber and the reaction chamber; and (b) a second fluid connection between the homogenization chamber and the reaction chamber.

In some embodiments of the system, the cartridge further comprises: (3) a filter assembly and a filter fluid path between the homogenization chamber and the filter assembly to obtain a filtered sample after the sample is homogenized in the homogenization chamber: and (4) a filtrate chamber for holding the filtered sample.

Another aspect of the present invention relates to a method for detecting the presence and/or absence of an molecule of interest in a sample comprising the steps of (a) collecting a sample suspected of containing an allergen of interest, (b) homogenizing the sample in an extraction buffer in the presence of a detection agent, thereby releasing the molecule of interest from the sample to engage in an interaction with the detection agent comprising a fluorescence moiety, (c) filtrating the homogenized sample containing the molecule of interest and the detection agent; (d) contacting the filtrate containing the molecule of interest and the detection agent with a detection probe molecule that engages in a probe interaction with the detection agent, wherein the interaction of the molecule of interest with the detection agent prevents the detection agent from engaging in the probe interaction with the detection probe; (e) washing off the contact in step (d) with wash buffer; (f) measuring signal outputs from the probe interaction of the detection probe molecule and the detection agent; and (g) processing and digitizing the detected signals and visualizing the interaction between the detection probe and the detection agent.

In some embodiments, the detection agent is an antibody or variant thereof, a nucleic acid molecule or a small molecule. In one preferred embodiment, the detection agent is a nucleic acid molecule comprising a nucleic acid sequence that binds to the molecule of interest and a fluorescence moiety attached to one end of the sequence. In some embodiments, the nucleic acid-based detection agent is stored in the buffer containing MgCl2.

In some embodiments, the detection probe molecule is a nucleic acid molecule that comprises a short nucleic acid sequence complementary to the sequence of the detection agent, wherein the probe molecule engages in a probe interaction with the detection agent and the interaction of the molecule of interest and the detection agent prevents the detection agent from engaging in the probe interaction.

In another aspect of the invention, a kit comprising a sample processing cartridge (e.g., a test cup as described herein), and instructions for use of the processing cartridge in testing for the presence of an allergen in a sample. In some embodiments, the kit may further comprise a sampler for collecting a sample.

In some embodiments, the detection system may comprise a user interface that may be accessed and controlled by a software application. The software may be run by a software application on a personal device such as a smartphone, a tablet computer, a personal computer, a laptop computer, a smartwatch and/or other devices. In some cases, the software may be run by an internet browser. In some embodiments, the software may be connected to a remote and localized server referred to as “the cloud.”

### BRIEF DESCRIPTION OF THE DRAWINGS

FIG. 1 is a perspective view of an embodiment of a detection system in accordance with the present invention comprising a detection device 100 having an external housing 101 and a port or receptacle 102 configured for holding the disposable cartridge 300, a separate food corer 200 as an example of the sampler, and a disposable test cup 300 as an example of the detection cartridge. Optionally, a lid 103 covers the receptacle 102. This embodiment of the system 100 has an execution/action button 104 that allows a user to execute an allergen detection testing and a USB port 105 may be included.

FIG. 2A is an exploded perspective view of one embodiment of the food corer 200 as an example of the sampler.

FIG. 2B is a perspective view of the food corer 200.

FIG. 3A is a perspective view of an embodiment of a disposable test cup 300, comprising a cup top 310, a cup body 320 and a cup bottom 330.

FIG. 3B is a cross-sectional view of the test cup 300, illustrating features inside the cup 300.

FIG. 3C is an exploded view of an embodiment of the disposable test cup 300.

FIG. 3D is a top (left panel) perspective view and a bottom (right panel) perspective view of the top cover 312.

FIG. 3E is a top perspective view (left panel) and a bottom perspective view (right panel) of the cup body 320.

FIG. 3F is a top perspective view of the bottom of the upper housing 320a (upper panel) shown in FIG. 3C and a bottom perspective view of the inside of the outer housing 320b (lower panel) shown in FIG. 3C.

FIG. 3G is a bottom perspective view (left panel) and a top perspective view (right panel) of the cup bottom cover 337 shown in FIG. 3C.

FIG. 3H is a bottom perspective view of the cup bottom surface after assembling the bottom 330 and the cup body 320.

FIG. 3I is an exploded view of the cup top lid 311.

FIG. 4A is an exploded view of one embodiment of the filter assembly 325.

FIG. 4B is a cross-sectional perspective view of one embodiment of the filtrate chamber 322 comprising a filter bed chamber 431 for placement of the filter assembly 325, a collection gutter 432 and a filtrate collection chamber 433.

FIG. 5A is a perspective view of an alternative embodiment of the test cup 300.

FIG. 5B is an exploded view of the disposable test cup 300 of FIG. 5A (the filter 325 is not shown).

FIG. 5C is a cross sectional elevation view of the cup 300 of FIG. 5A.

FIG. 5D is an exploded perspective view of an alternative embodiment of the test cup 300.

FIG. 5E is a bottom perspective view (upper panel) and a top perspective view (bottom panel) of the cup body 320 shown in FIG. 5D.

FIG. 5F is a bottom perspective view of the cup bottom 337 and the bottom of the cup body 320 shown in FIG. 5D.

FIG. 5G is an alternative embodiment of the filter assembly 525.

FIG. 5H is a cross-sectional view of the filter cap 541 of the filter assembly 525 when assembled together with the valve 350.

FIG. 5I includes a perspective view of the rotary valve 350 (upper panel), a side elevation view of the rotary valve 350 (lower left panel) and a bottom view of the bottom of the rotary valve 350 (lower right panel).

FIG. 5J is a bottom view (upper panel) of the cup bottom cover 337 and a top view (lower panel) of the cup bottom cover 337 shown in FIG. 5D.

FIG. 5K is a top view of the chip panel 532 shown in FIG. 5D.

FIG. 6A is a top view of the upper cup body 510 showing features relating to homogenization, filtration (F), wash (W1 and W2) and waste.

FIG. 6B is a scheme showing the positions of the rotary valve 350 during the sample preparation and sample washes.

FIG. 6C is a diagram displaying the fluid flow inside the cup 300.

FIG. 7A is a perspective view of the device 100.

FIG. 7B is a top view of the device 100 in the absence of the lid 103.

FIG. 8A is a longitudinal cross-sectional perspective view of the device 100.

FIG. 8B is a lateral cross-sectional perspective view of the device 100.

FIG. 9A is a valve motor 820 and associated components for controlling the operation of the rotary valve 350.

FIG. 9B is a top perspective view of the output coupling 920 associated with the motor.

FIG. 10A is a top perspective view of one embodiment of the optical system 830.

FIG. 10B is a side view of the optical system 830 of FIG. 10A.

FIG. 11A is an illustration of a chip sensor 333 displaying the test area and control areas.

FIG. 11B is a top view of the optical system 830 and chip 333 showing reflections providing fluorescence measurements of the chip 333.

FIG. 12A shows the optical assembly 830 in a straight mode.

FIG. 12B shows the optical assembly 830 in a folded mode.

FIG. 12C is a cross-sectional perspective view of one end of the device 100 (right side of FIG. 8B) showing emission optics 1210 including lenses 1221, 1223 and filters 1222a and 1222b placed in the stepped bore 1224 in the device 100.

FIG. 13A is a histogram demonstrating the SPN intensity in a MgCl₂-lyophilized formulation as compared to the buffer without MgCl₂ and the MgCl₂ solution.

FIG. 13B shows the percentage of magnesium recovered from MgCl₂ formulations deposited on the cotton filter supported on 1 μm mesh.

### DETAILED DESCRIPTION OF THE INVENTION

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. Unless defined otherwise, all technical and scientific terms used herein have the same meaning as commonly understood by one of ordinary skill in the art to which this invention belongs. In the case of conflict, the present description will control.

The use of analytical devices to ensure food safety has not yet advanced to the point of fulfilling its promise. In particular, portable devices based on simple, yet accurate, sensitive and rapid detection schemes have not yet been developed for detection of a wide variety of known allergens. One of the more recent reviews of aptamer-based analysis in context of food safety control indicated that while a great variety of commercial analytical tools have been developed for allergen detection, most of them rely on immunoassays. It was further indicated that the selection of aptamers for this group of ingredients is emerging (Amaya-Gonzalez et al., Sensors 2013, 13, 16292-16311, the contents of which are incorporated herein by reference in their entirety).

The present invention provides detection systems and devices that can specifically detect low concentrations of allergens in a variety of food samples. The detection system and/or device of the present invention is a miniaturized, portable and hand-held product, which is intended to have a compact size which enhances its portability and discreet operation. A user can carry the detection system and device of the present invention and implement a rapid and real-time test of the presence and/or absence of one or more allergens in a food sample, prior to consuming the food. The detection system and device, in accordance with the present invention, can be used by a user at any location, such as at home or in a restaurant. The detection system and/or device displays the test result as a standard readout and the detection can be implemented by any user following the simple instructions on how to operate the detection system and device.

In some embodiments, the detection system and device is constructed for simple, fast, and sensitive one-step execution. The system may complete an allergen detection test in less than 5 minutes, or less than 4 minutes, or less than 3 minutes, or less than 2 minutes, or less than 1 minute. In some examples, the allergen detection may be completed in approximately 60 seconds, 55 seconds, 50 seconds, 45 seconds, 40 seconds, 35 seconds, 30 seconds, 25 seconds, 20 seconds, or 15 seconds.

In accordance with the present invention, the construction process for producing the detection system and device may be a mechatronic construction process integrating electrical engineering, mechanical engineering and computing engineering to implement and control the process of an allergen detection test. Embodiments of the detection system and device have features including but not limiting to rechargeable or replaceable batteries, motor drivers for processing the test sample, pumps for controlling the flow of the processed sample solutions and buffers within the cartridge, printed circuit boards and connectors that couple and integrate different components for a fast allergen testing. Embodiments of the detection device of the present invention also include an optical system which is configured for detection of the presence and concentration of an allergen of interest in a test sample and conversion of detection signals into readable signals; and a housing which provides support for other parts of the detection device and integrates different parts together as a functional product.

In some embodiments, the detection system and/or device is constructed such that the disposable detection cartridges (e.g., a disposable test cup or cup-like container), unique to one or more specific allergens, are constructed for receiving and processing a test sample and implementing the detection test, in which all the solutions are packed. Therefore, all the solutions may be confined in the disposable cup or cup-like container. As a non-limiting example, a disposable peanut test cup may be used to detect peanut in any food sample by a user and discarded after the test. This prevents cross-contamination when different allergen tests are performed using the same device.

In some embodiments, a separate sampler that can measure and size a test sample is provided. In one aspect, the sampler can further pre-process the test sample, such as cutting the sample into small pieces, blending, abrading and/or grinding, to make the sample suitable for allergen protein extraction.

In accordance with the present invention, nucleic acid molecules (i.e., aptamers) that specifically bind to an allergen of interest in a sample are used as detection agents. The nucleic acid agents may be aptamers and signaling polynucleotides (SPNs) derived from aptamers that can recognize the target allergen. In some embodiments, the SPNs capture the allergen proteins in the sample to form SPN:protein complexes. Another detection probe such as short nucleic acid sequences that are complementary to the SPN sequence may be used to anchor the SPN to a solid substrate for signal detection. In other embodiments, the detection agents may be attached to a solid substrate such as the surface of a magnetic particle, silica, agarose particles, polystyrene beads, a glass surface, a microwell, a chip (e.g., a microchip), or the like. It is within the scope of the present invention that such detection agents and sensors can also be integrated into any suitable detection systems and instruments for similar purposes.

The aptamers and SPNs that specifically bind to a target allergen may be those disclosed in commonly owned U.S. Provisional Application Ser. No. 62/418,984, filed on Nov. 8, 2016; 62/435,106, filed on Dec. 16, 2016; and 62/512,299, filed on May 30, 2017; and the PCT patent application Publication No. WO/2018/089391, filed on Nov. 8, 2017; the contents of each of which are incorporated herein by reference in their entirety.

### Detection Systems

According to the present invention, an allergen detection system of the present invention may comprise at least one disposable detection cartridge for implementing an allergen detection test, and a detection device for detecting and visualizing the result of the detection test. Optionally the detection system may further comprise at least one sampler for collecting a test sample. The sampler can be any tool that can be used to collect a portion of a test sample, e.g., a spoon or a chopstick. In some aspects, a particularly designed sampler may be included to the present detection system as discussed hereinbelow.

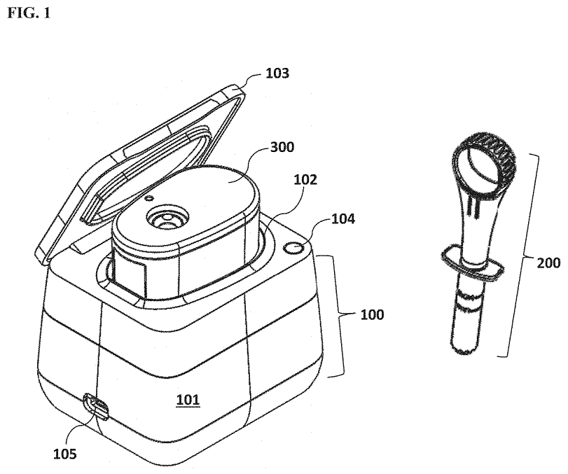

As shown in FIG. 1, an embodiment of the detection system of the present invention comprises a detection device 100 configured for processing a test sample, implementing an allergen detection test, and detecting the result of the detection test, a separate food corer 200 as an example of the sampler, and a disposable test cup 300 as an example of the detection cartridge. The detection device 100 includes an external housing 101 that provides support to the components of the detection device 100. A port or receptacle 102 of the detection device 100 is constructed for docking the disposable test cup 300 and a lid 103 is included to open and close the instrument. The external housing 101 also provides surface space for buttons that a user can operate the device. An execution/action button 104 that allows a user to execute an allergen detection testing and a USB port 105 may be included. Optionally a power plug (not shown) may also be included. During the process of implementing an allergen detection test, the food corer 200 with a sample contained therein is inserted into the disposable test cup 300 and the disposable test cup 300 is inserted into the port 102 of the detection device 100 for detection.

### Sampler

Collecting an appropriately sized sample is an important step for implementing allergen detection testing. In some embodiments of the present invention, a separate sampler for picking up and collecting test samples (e.g. food samples) is provided. In one aspect, a coring-packer-plunger concept for picking up and collecting a food sample is disclosed herein. Such mechanism may measure and collect one or several sized portions of the test sample and provide pre-processing steps such as cutting, grinding, abrading and/or blending, for facilitating the homogenization and extraction or release of allergen proteins from the test sample. According to the present invention, a separate food corer 200 is constructed for obtaining different types of food samples and collecting an appropriately sized portion of a test sample.

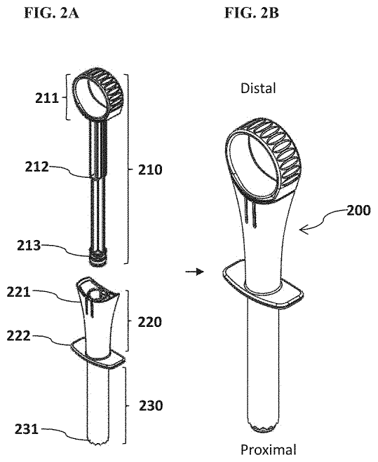

As shown in FIG. 2A, the food corer 200 may comprise three parts: a plunger 210 at the distal end, a handle 220 configured for coupling a corer 230 at the proximal end. The plunger 210 has a distal portion provided with a corer top grip 211 (FIG. 2A) at the distal end, which facilitates maneuverability of the plunger 210 up and down, a plunger stop 212 in the middle of the plunger body, and a seal 213 at the proximal end of the plunger body. The handle 220 may comprise a snap fit 221 at the distal end and a skirt 222 at the proximal end connecting to the corer 230. The corer 230 may comprise a proximal portion provided with a cutting edge 231 at the very proximal end (FIG. 2A). The corer 230 is configured for cutting and holding the collected sample to be expelled into the disposable test cup 300.

In one embodiment, the plunger 210 may be inserted inside the corer 230, where the proximal end of the plunger 210 may protrude from the corer 230 for directly contacting a test sample, and together with the cutting edge 231 of the corer 230, picking up a sized portion of the test sample (FIG. 2B). In accordance with the present invention, the plunger 210 is used to expel sampled food from the corer 230 into the disposable test cup 300, and to pull certain foods into the corer 230 as well, such as liquids and creamy foods. The feature of the plunger stop 212, through an interaction with the snap fit 221, may prevent the plunger 210 from being pulled back too far or out of the corer body 230 during sampling. The seal 213 at the very proximal end of the plunger 210 may maintain an air-tight seal in order to withdraw liquids into the corer 230 by means of pulling the plunger 210 back. In some embodiments, the plunger 210 may be provided with other types of seals including a molded feature, or a mechanical seal. The handle 220 is constructed for a user to hold the coring component of the sampler 200. For example, the skirt 222 gives the user means for operating the food sampler 200, pushing down the corer 230 and driving the corer 230 into the food sample to be collected.

In some embodiments, the cutting edge 231 may be configured for pre-processing the collected sample, allowing the sampled food to be cored in a pushing, twisting and/or cutting manner. As some non-limiting examples, the cutting edge 231 may be in a flat edge, a sharp edge, a serrated edge with various numbers of teeth, a sharp serrated edge and a thin wall edge. In other aspects, the inside diameter of the corer 230 varies, ranging from about 5.5 mm to 7.5 mm. Preferably the inside diameter of the corer 230 may be from about 6.0 mm to about 6.5 mm. The inside diameter of the corer 230 may be 6.0 mm, 6.1 mm, 6.2 mm, 6.3 mm, 6.4 mm, 6.5 mm, 6.6 mm, 6.7 mm, 6.8 mm, 6.9 mm, or 7.0 mm. The size of the corer 230 is optimized for a user to collect a right amount of the test sample (e.g., 1.0 g to 0.5 g).

The parts of the food corer 200 may be constructed as any shape for easy handling such as triangular, square, octagonal, circular, oval, and the like.

In other embodiments, the food corer 200 may be further provided with a means for weighing a test sample being picked up, such as a spring, a scale or the equivalent thereof. As a non-limiting example, the food corer 200 may be provided with a weigh tension module.

The food corer 200 may be made of plastic materials, including but not limited to, polycarbonate (PC), polystyrene (PS), poly(methyl methacrylate) (PMMA), polyester (PET), polypropylene (PP), high density polyethylene (HDPE), polyvinylchloride (PVC), thermoplastic elastomer (TPE), thermoplastic urethane (TPU), acetal (POM), polytetrafluoroethylene (PTFE), or any polymer, and combinations thereof.

The sampler (e.g., the corer 200) may be operatively associated with an analytical cartridge (e.g., the disposable cup 300) and/or a detection device (e.g. the device 100). Optionally, the sampler may comprise an interface for connecting to the cartridge. Optionally, a cap may be positioned on the proximal end of the sampler. The sampler 200 may also comprise a sensor positioned with the sampler 200 to detect a presence of a sample in the sampler.

### Disposable Processing Cartridge

In some embodiments, the present invention provides a detection cartridge or vessel. As used herein, the terms “cartridge” and “vessel’ are used interchangeably. The cartridge is constructed for implementing a detection test. The detection cartridge is disposable and used for a particular allergen. A disposable detection cartridge is constructed for dissociation of food samples and allergen protein extraction, filtration of food particles, storage of reaction solutions/reagents and detection agents, and capture of an allergen of interest using detection agents such as antibodies and nucleic acid molecules that specifically bind to allergen proteins. In one aspect, the detection agents are nucleic acid molecules such as aptamers and/or aptamer-derived SPNs. In other embodiments, the detection agents may be antibodies specific to allergen proteins, such as antibodies specific to peanut allergen protein Ara H1. In accordance with the present invention, at least one separate detection cartridge is provided as part of the detection system. In other embodiments, the detection cartridge may be constructed for use in any other detection systems.

In some embodiments, the detection cartridge may be constructed to comprise one or more separate chambers, each configured for separate functions such as sample reception, protein extraction, filtration, and storage for buffers, agents and waste solution. The detection cartridge may also comprise a means for processing the sample (e.g., a homogenizer), a filter for filtering off large particles and channels and ports for controlling the fluid flow inside the cartridge.

In some embodiments, a disposable detection cartridge is intended to be used only once for an allergen test in a sample and therefore may be made of low cost plastic materials, for example, acrylonitile butadiene styrene (ABS), COC (cyclic olefin copolymer), COP (cyclo-olefin polymer), transparent high density polyethylene (HDPE), polycarbonate (PC), poly(methyl methacrylate) (PMMA), polypropylene (PP), polyvinylchloride (PVC), polystyrene (PS), polyester (PET), or other thermoplastics. Accordingly, a disposable detection cartridge may be constructed for any particular allergen of interest. In some embodiments, these disposable cartridges may be constructed for one particular allergen only, which may avoid cross contamination with other allergen reactions.

In some embodiments, the disposable cartridge is made of polypropylene (PP), COC (cyclic olefin copolymer), COP (cyclo-olefin polymer), PMMA (poly(methyl methacrylate), or acrylonitrile butadiene styrene (ABS).

In other embodiments, these disposable cartridges may be constructed for detecting two or more different allergens in a test sample in parallel. In some aspects, the disposable cartridges may be constructed for detecting two, three, four, five, six, seven, or eight different allergens in parallel. In one aspect, the presence of multiple allergens, e.g., two, three, four, five, or more, are detected simultaneously, a positive signal may be generated indicating which allergen is present. In another aspect, a system is provided to detect if an allergen, e.g., peanut or a tree-nut, is present and generate a signal to indicate the presence of such allergen.

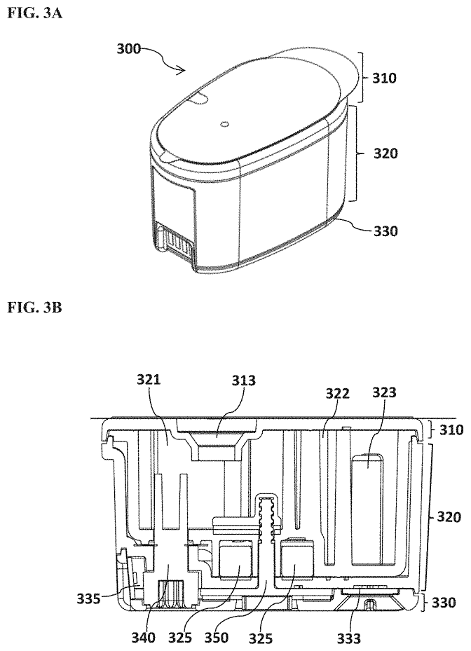

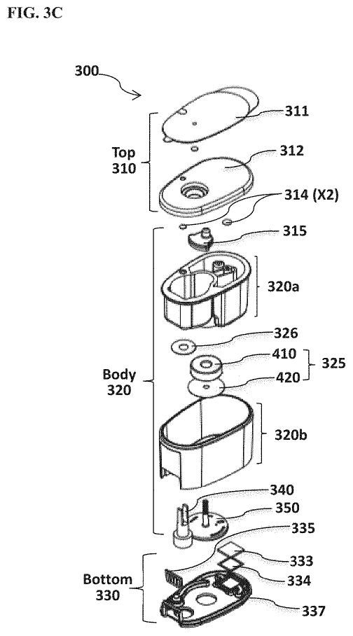

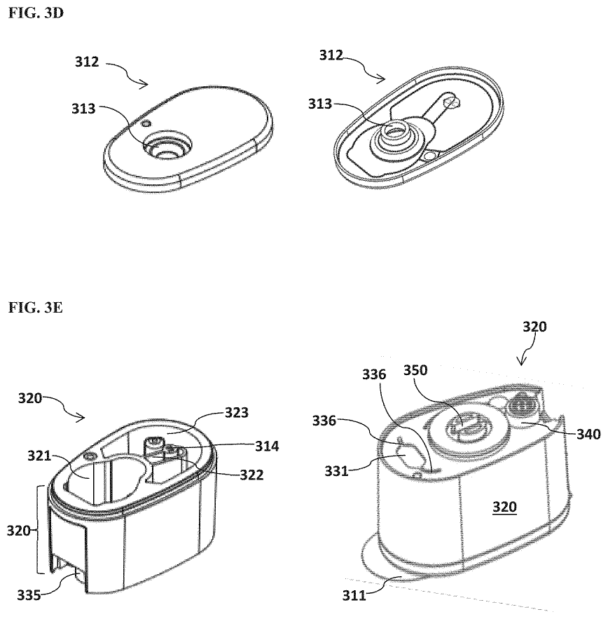

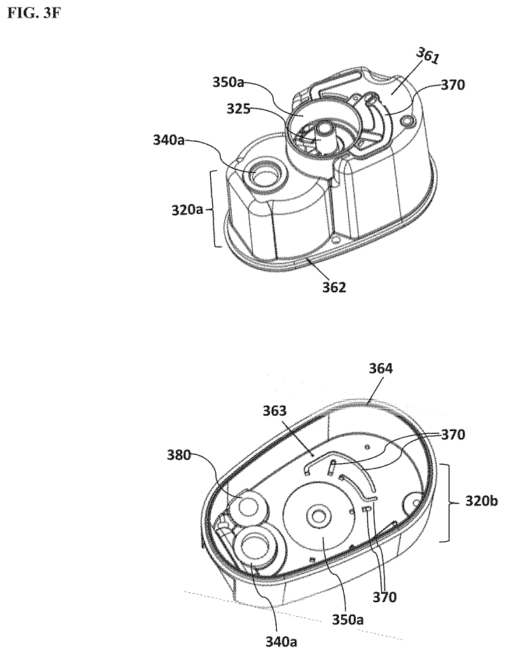

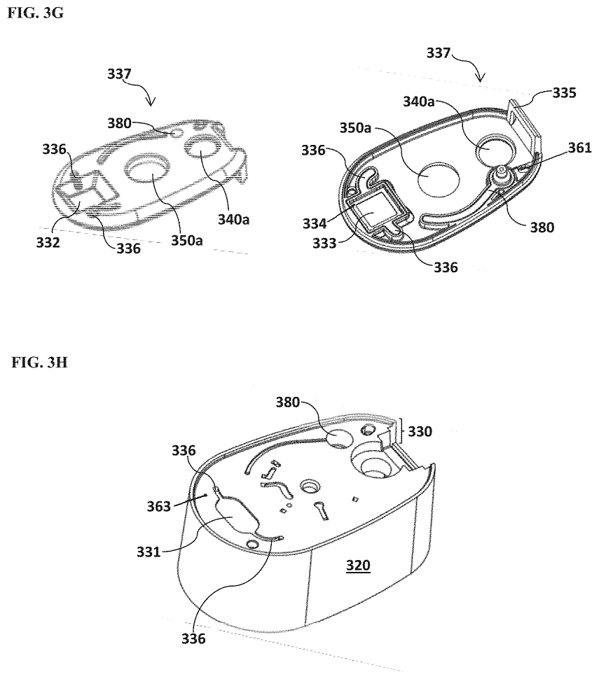

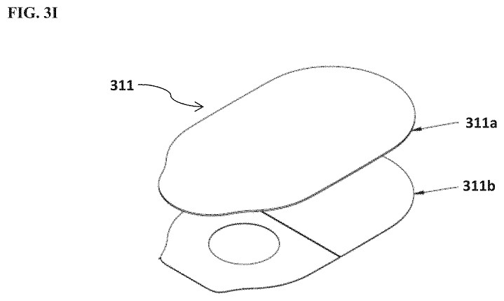

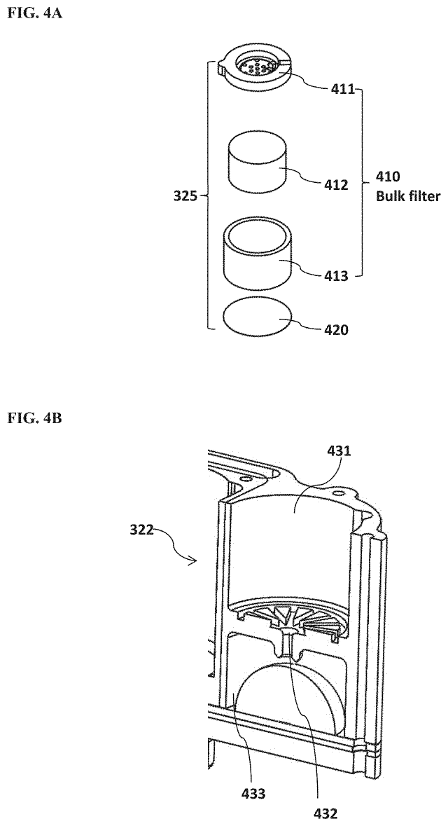

In some embodiments, the disposable detection cartridge may be a disposable test cup or a cup-like container. According to one embodiment of the test cup, as shown in FIG. 3A, the assembled disposable test cup 300 comprises three parts: a cup top 310, a cup body 320 and a cup bottom 330. The cup 300 further comprises a homogenization rotor 340 that rotates in both directions to homogenize the sample, and a rotary valve 350 for fluid flow inside the cup (FIG. 3B).

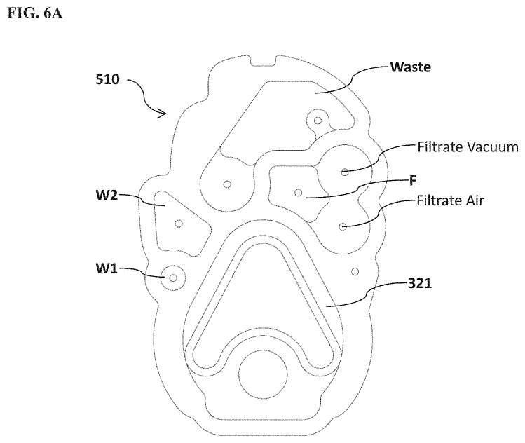

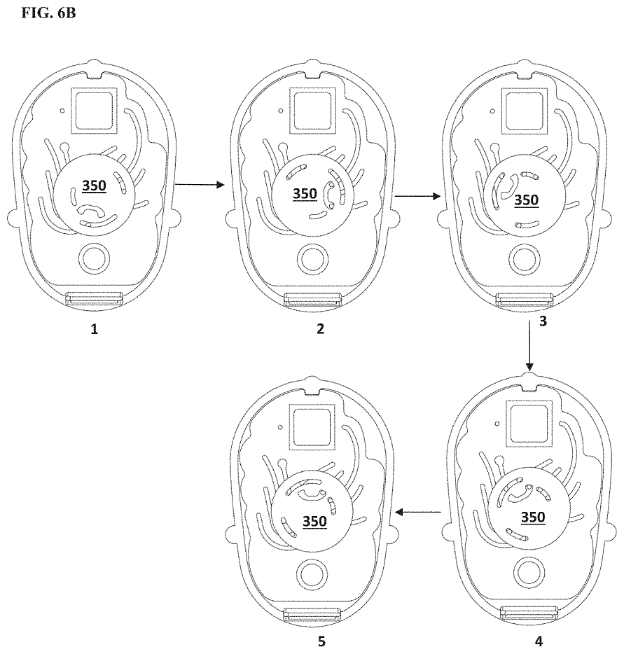

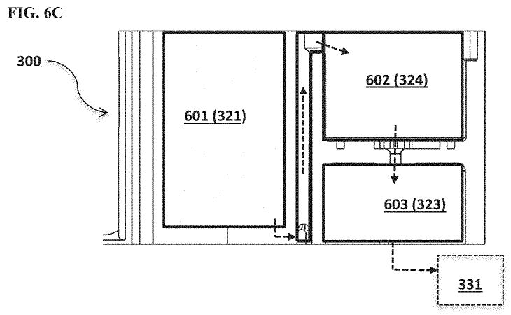

In some embodiments, the test cup body 320 may include a plurality of chambers. In one embodiment, as shown in FIG. 3B, the test cup body 320 includes one homogenization chamber 321 comprising a food processing reservoir 601 (as shown in FIG. 6C), a filtrate chamber 322 for collecting a sample solution after being filtered through the filter (e.g., the 2-state filter 325), a waste chamber 323 comprising a waste reservoir 603 (as shown in FIG. 6C), and optionally, a wash buffer storage chamber 324 comprising wash buffer storage reservoir 602 (as shown in FIG. 6C). A reaction chamber 331 at the cup bottom 320 (also referred to herein as a signal detection chamber) is shown in FIGS. 3E and 3H. All analytical reactions occur in the reaction chamber 331, and a detectable signal (e.g., a fluorescence signal) is generated therein. In some embodiments, detection agents (e.g., SPNs) for example, which are pre-stored in the homogenization chamber 321, may be premixed with the test sample in the homogenization chamber 321, where the test sample is homogenized and the extracted allergen proteins react with the detection agents. The mixed reaction complexes may be transported to the filter 325 before they are transported to the reaction chamber 331, wherein the detection signal is measured.

In alternative embodiments, more than one buffer and reagent storage reservoir may be included in the buffer and reagent storage chamber 324. As a non-limiting example, the extraction buffer and wash buffers may be stored separately in a reservoir within the buffer storage chamber 324.

FIG. 3C shows an exploded view of the disposable test cup 300 which is configured to contain three main components, the top 310, the housing or body 320 and the bottom 330. In one embodiment, the cup top 310 may include a cup lid 311, a top cover 312 having a food corer port 313 (in FIGS. 3B and 3D) for receiving a food corer 200, two or more breather filters 314 which are included to ensure that only air is brought in and that fluids do not escape from the test cup 300. The top part may have two lids 311. As shown in FIG. 3I, the second lid at the bottom 311b is constructed for resealing and liquid retention during the operation. The top lid 311a may be peeled back for inserting the test sample collected by the corer 200, and then reclosed after assay completion. The top cover 312 may also include at least one small hole (FIG. 3C) for air to be drawn in for fluid flow. The cup body 320 is composed of two separate parts: an upper housing 320a and an outer housing 320b. A filter or filter assembly 325 is included in the cup body for processing the sample. The filter 325 may be attached to the cup body through the gasket 326. The cup bottom assembly 330 includes a bottom cover 337 that sandwiches other components including the reaction chamber 331 (in FIGS. 3E and 3G), a detection sensor, i.e. a glass chip 333, and a chip gasket 334 that facilitates the attachment of the glass chip 333 to the bottom of the reaction chamber 331. The bottom cover 337 also comprises a port/bit 340a for holding the homogenization rotor 340 and a port/bit 350a for holding the rotary valve 350 (as shown in FIG. 3G). These bits provide a means for linking the homogenization rotor 340 and the rotary valve 350 to the motors of the detection device 100. For example, a rotor gasket may be configured to the upper housing 320a to seal the rotor 340 to the housing 320, to avoid leakage of fluids.

In some embodiments, the cup may further be constructed to comprise a bar code that can store lot-specific parameters. In one example, the bar code may be the data chip 335 that stores the cup 300 specific parameters, including the information of SPNs (e.g., fluorophore labels, the target allergen, and intensity of SPNs, etc.), expiration date, manufacture information, etc.

FIG. 3D further demonstrates the features of the top cover 312 of the cup shown in FIG. 3A. A corer port 313 is included for receiving the sampler and transferring the picked test sample to the sample processing chamber 321. As a non-limiting example, the port 313 may be configured for receiving the food corer 200 shown in FIG. 2B. FIG. 3E is a top perspective view of a cup housing body 320. The upper housing 320a and the outer housing 320b shown in FIG. 3C are assembled together in this view. The upper housing 320a may comprise one or more chambers which are operatively connected. In this embodiment, the homogenization chamber 321, filtration chamber 322 and waste chamber 323 can be seen (left panel). The bottom of the cup body 320 comprises the reaction chamber 331 with the inlet and outlet 336 for fluid flow (right panel). The rotor 340 and the rotary valve 350 may be assembled in the cup 300 to form a functional detection cartridge (right panel).

FIG. 3F further illustrates the outer interface of the bottom of the upper housing (320a shown in FIG. 3C) (upper panel) and the inner interface of the bottom of the outer housing 320b shown in FIG. 3C (lower panel). The two energy-director faces 361 (face 1) and 362 (face 2) at the outer interface of the upper housing 320a, interact with the two welding mating faces, face 363 (face 1) and 364 (face 2) at the inner interface of the bottom of the outer housing 320b to retain the housing parts 320a and 320b together to form the cup body 320. Fluid paths 370 are also included to flow liquids in the cup bottom 330. The rotor 340 and the rotary valve 350 are assembled into the cup 300 through the rotor port 340a and the rotary valve port 350a, respectively.

FIG. 3G further illustrates the bottom cover 337 of the cup 300 shown in FIG. 3A and FIG. 3C. After the parts are assembled together to form a functional test cup 300, a specialized area 332 within the reaction chamber 331 may comprise a detection sensor that comprises detection agents such as SPNs specific to the allergen to be detected. In one embodiment, the detection sensor is the glass chip 333 is positioned to the reaction area 332 through a glass gasket 334 (as shown in FIG. 3C). The glass gasket 334 may be included to seal the glass chip 333 in place at the bottom of the reaction area 332 of the reaction chamber 331 and to prevent fluid leakage. Alternatively, adhesive or ultrasonic bonding can be used to mate the layers together. In some aspects of the present invention, the glass chip 333 may be configured directly at the bottom of the reaction chamber 331 (e.g., the bottom surface of the sensor area 332) as a component of the cup bottom cover 337) and integrated into the cup body 320 as one entity. The entire unit may be constructed of PMMA (poly(methyl methacrylate)) (also referred to as acrylic or acrylic glass). This transparent PMMA acrylic glass may be used as an optical window for signal detection.

As shown in FIG. 3H, the bottom 330 is assembled together with the cup body 320. From this bottom perspective view, the bottom surface of the cup comprises several interfaces for fluid paths (e.g., fluidic inlet/outlet 336) and a pump interface 380 and the interfaces connecting the rotor 340 and the rotary valve 350 shown in FIG. 3C to the detection device 100.

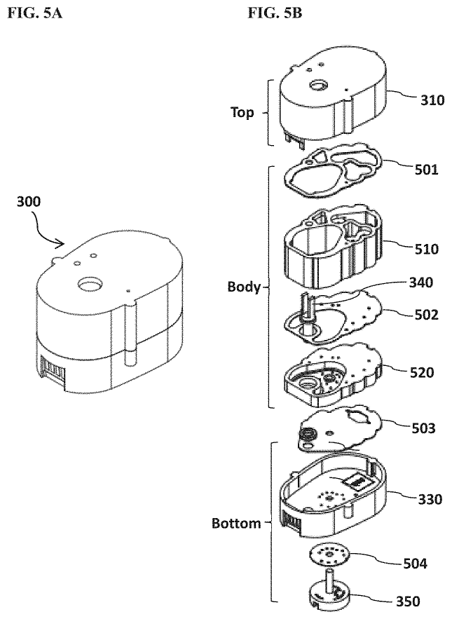

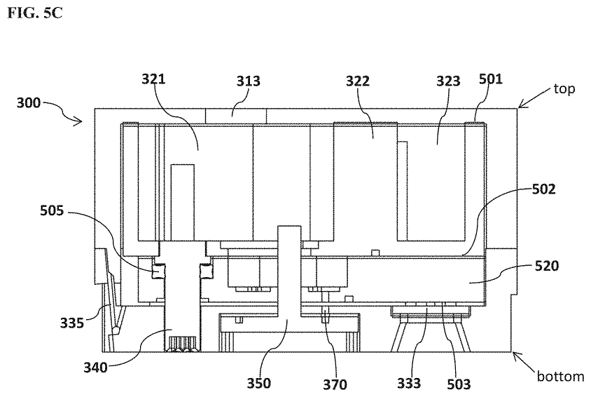

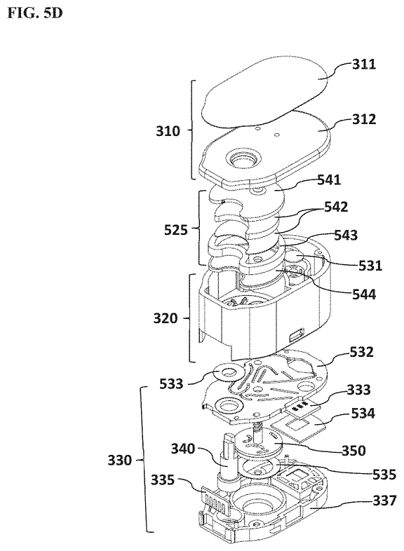

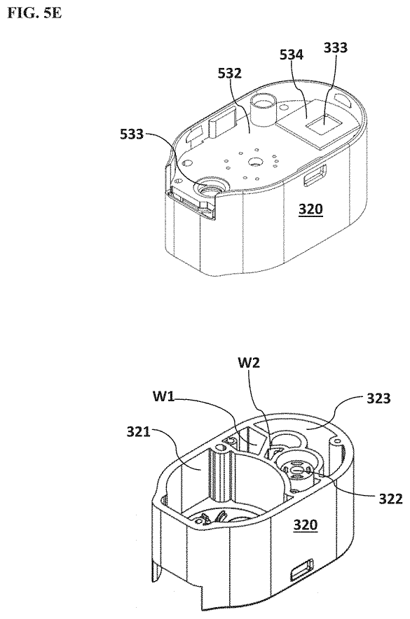

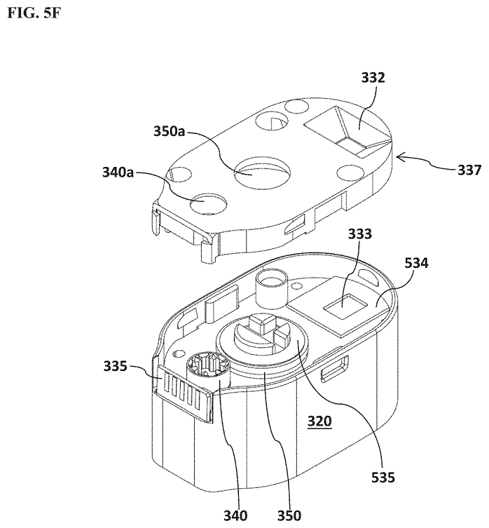

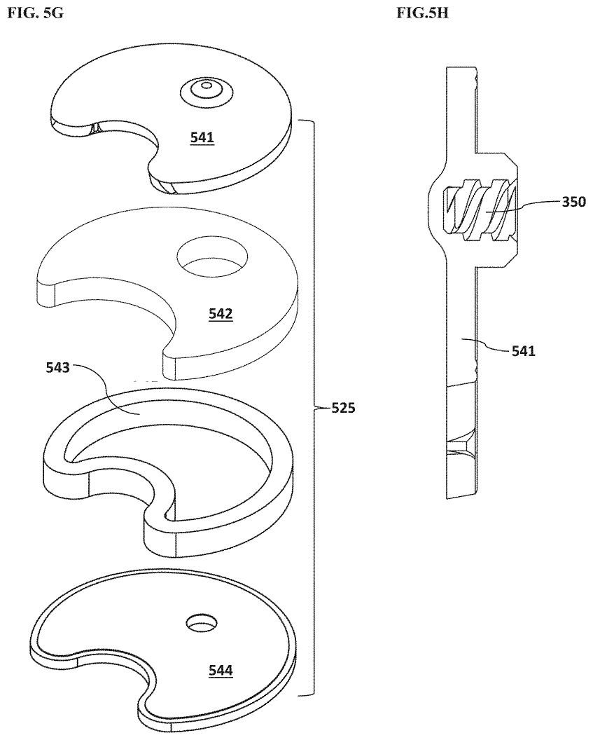

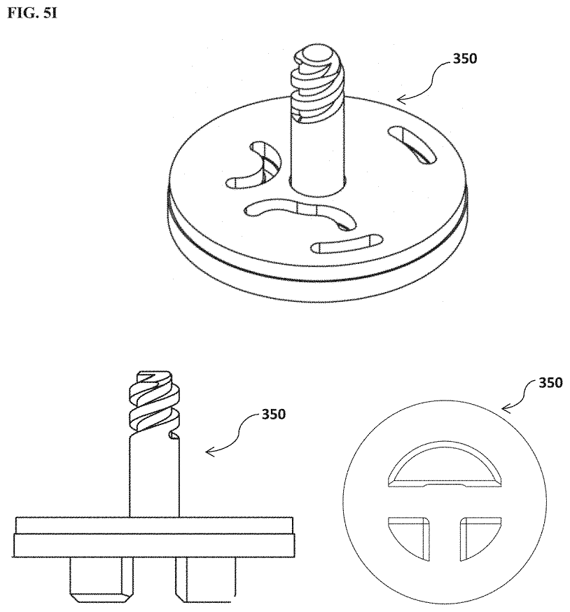

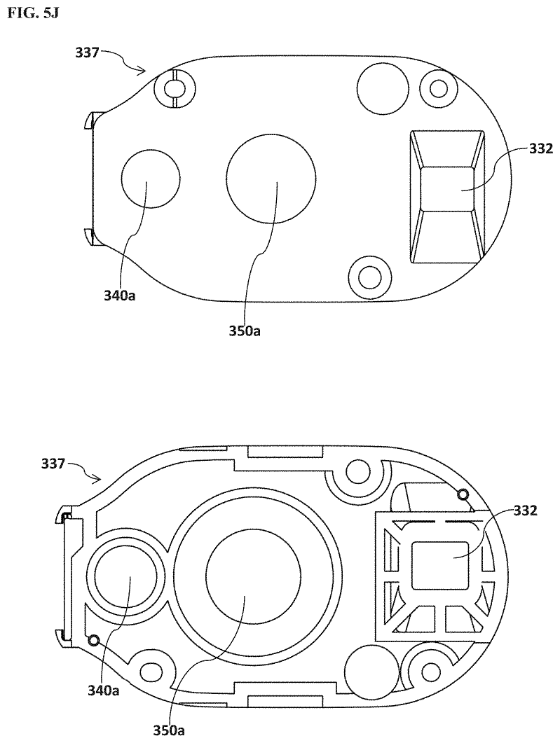

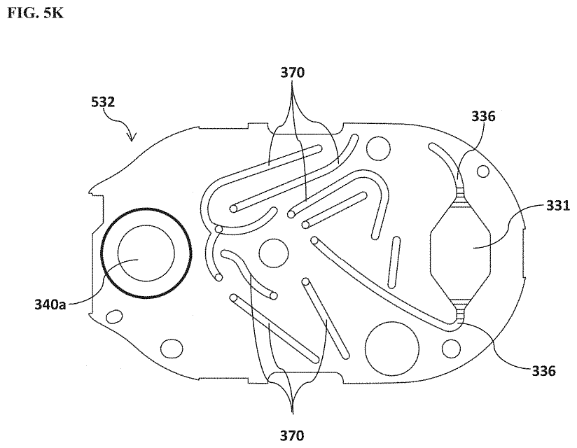

A means may be included in the cup 300 to block the flow of fluids between the compartments of the cup 300. In one embodiment, a dump valve 315 (in FIG. 3C) in the cup housing 320a is included to block fluid in homogenization chamber 321 from flowing to the rotary valve 350 that is configured at the bottom of the cup 300. The dump valve 315 is held in place by the rotary valve 350 for shipping and end of life. The rotary valve 350 locks the dump valve 315 over the filters (e.g., the filter assembly 325) during shipping and prevents fluid flow after completing the detection assay. In some embodiments, the rotary valve 350 may comprise a valve shaft that is operatively connected to and locks the dump valve 315 (as shown in FIG. 3C). The rotary valve 350 can be attached to the cup 300 through any available means known in the art. In one embodiment, a valve gasket (e.g., the gasket 504 shown in FIG. 5A) may be used. Alternatively, the rotary valve 350 can be attached to the cup through a wave disc spring. The rotary valve 350 may be actuated in several steps to direct flow to the proper chambers inside the cup 300. As a non-limiting example, the positions of the rotary valve 350 during the detection test are demonstrated in FIG. 6B.

In some embodiments, a filter assembly (e.g., the filter 325 shown in FIG. 3C and FIG. 4A) is included into the cartridge for removing large particles and other interfering components from the sample, such as fat from a food matrix, before the processed sample is transferred into the reaction chamber 331.

In some embodiments, the filter mechanism may be a filter assembly. The filter assembly may be a simple membrane filter 420. The membrane 420 may be a nylon, PE, PET, PES (poly-ethersulfone), Porex™, glass fiber, or the membrane polymers such as mixed cellulose esters (MCE), cellulose acetate, PTFE, polycarbonate, PCTE (polycarbonate) or PVDF (polyvinylidene difluoride), or the like. It may be a thin membrane (e.g., 150 μm thick) with high porosity. In some embodiments, the pore size of the filter membrane 420 may range from 0.01 μm to 600 μm, or from 0.1 μm to 100 μm, or from 0.1 μm to 50 μm, or from 1 μm to 20 μm, or from 20 μm to 100 μm, or from 20 μm to 300 μm, or 100 μm to 600 μm or any size in between. For example, the pore size may be about 0.02 μm, about 0.05 μm, about 0.1 μm, about 0.2 μm, about 0.5 μm, about 1.0 μm, about 1.5 μm, about 2.0 μm, about 2.5 μm, about 3 μm, about 3.5 μm, about 4.0 μm, about 4.5 μm, about 5.0 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 100 μm, about 150 μm, about 200 μm, about 250 μm, about 300 μm, about 350 μm, about 400 μm, about 450 μm, about 500 μm, about 550 μm, or about 600 μm.

In some alternative embodiments, the filter assembly may be a complex filter assembly 325 (as shown in FIG. 4A) comprising several layers of filter materials. In one example, the filter assembly 325 may comprise a bulk filter 410 composed of a gross filter 411, a depth filter 412, and a membrane filter 420 (FIG. 4A). In one embodiment, the gross filter 411 and the depth filter 412 may be held by a retainer ring 413 to form a bulk filter 410 sitting on the membrane filter 420. In other embodiments, the bulk filter 410 may further comprise a powder that sits inside the filter or on top of the filter. The powder may be selected from cellulose, PVPP, resin, or the like. In some examples, the powder does not bind to nucleic acids and proteins.

In some embodiments, the filter assembly 325 may be optimized for removing oils from highly fatty samples, but not proteins and nucleic acids, resulting in superior sample cleaning. In other embodiments, the ratio of the depth and width of the filter assembly 325 may be optimized to maximize the filtration efficiency.

In some embodiments, the filter assembly 325 may be placed inside a filter bed chamber 431 (FIG. 4B) in the disposable cup body 320. The filter bed chamber 431 may be connected to the homogenization chamber 321. The homogenate can be fed to the filter assembly 325 inside the filter bed chamber 431. The filtrate is collected by the collection gutter 432 (also referred to herein as filtrate chamber). The collected filtrate then can exit the fluidics to flow to the reaction chamber 331 (FIG. 3B). In one example, the collected filtrate may be transported to the reaction chamber 331 from the collection gutter 432 directly. In another example, the filtrate may be first transported to the filtrate collection chamber 433 before being transported to the reaction chamber 331 through the inlet/outlet 336 (FIG. 3G). The fluids may be delivered to the reaction chamber 331 by the fluid paths 370 at the bottom of the cup 320 (as shown in FIG. 3F).

In some embodiments, the filtrate collection chamber 433 may further comprise a filtrate concentrator which is configured to concentrate the sample filtrate before it flows to the reaction chamber 331 for signal detection. The concentrator may be in a half-ball shape, or a conical type concentrator, or a tall pipe.

In accordance with this embodiment, the processed sample (e.g., the homogenate from the chamber 321) is filtered sequentially through the gross filter 411, the depth filter 412 and the membrane filter 420. The gross filter 411 can filter a large particle suspension from the sample, for example, particles larger than 1 mm. The depth filter 412 may remove small particle collections and oil components from the sample (such as the food sample). The pore size of the depth filter 412 may range from about 1 μm to about 500 μm, or about 1 μm to about 100 μm, or about 1 μm to about 50 μm, or about 1 μm to about 20 μm, or about 4 μm to about 20 μm, or from about 4 μm to about 15 μm. For example, the pore size of the depth filter 412 may be about 2 μm, or about 3 μm, or about 4 μm, or about 5 μm, or about 6 μm, or about 7 μm, or about 8 μm, or about 9 μm, or about 10 μm, or about 11 μm, or about 12 μm, or about 13 μm, or about 14 μm, or about 15 μm, or about 16 μm, or about 17 μm, or about 18 μm, or about 19 μm, or about 20 μm, or about 25 μm, or about 30 μm, or about 35 μm, or about 40 μm, or about 45 μm, or about 50 μm.

The depth filter 412 may be composed of, for example, cotton including, but not limited to raw cotton and bleached cotton, polyester mesh (monofilament polyester fiber) or sand (silica). In some embodiments, the filter material may be hydrophobic, hydrophilic or oleophobic. In some examples, the material does not bind to nucleic acids and proteins. In one embodiment, the depth filter is a cotton depth filter. The cotton depth filter may vary in sizes. For example, the cotton depth filter may have a ratio of width and height ranging from about 1:5 to about 1:20. The cotton depth filter 412 may be configured to correlate total filter volume and the food mass being filtered.

The membrane filter 420 can remove small particles less than 10 μm in size, or less than 5 μm in size, or less than 1 μm in size. The pore size of the membrane may range from about 0.001 μm to about 20 μm, or from 0.01 μm to about 10 μm. Preferably the pore size of the filter membrane may be about 0.001 μm, or about 0.01, or about 0.015 μm, or about 0.02 μm, or about 0.025 μm, or about 0.03 μm, or about 0.035 μm, or about 0.04 μm, or about 0.045 μm, or about 0.05 μm, or about 0.055 μm, or about 0.06 μm, or about 0.065 μm, or about 0.07 μm, or about 0.075 μm, or about 0.08 μm, or about 0.085 μm, or about 0.09 μm, or about 0.095 μm, or about 0.1 μm, or about 0.15 μm, or about 0.2 μm, or about 0.2 μm, or about 0.25 μm, or about 0.3 μm, or about 0.35 μm, or about 0.4 μm, or about 0.45 μm, or about 0.5 μm, or about 0.55 μm, or about 0.6 μm, or about 0.65 μm, or about 0.7 μm, or about 0.75 μm, or about 0.8 μm, or about 0.85 μm, or about 0.9 μm, or about 1.0 μm, or about 1.5 μm, or about 2.0 μm, or about 3.0 μm, or about 3.5 μm, or about 4.0 μm, or about 4.5 μm, or about 5.0 μm, or about 6.0 μm, or about 7.0 μm, or about 8.0 μm, or about 9.0 μm, or about 10 μm. As discussed above, the membrane may be a nylon membrane, PE, PET, a PES (poly-ethersulfone) membrane, a glass fiber membrane, a polymer membrane such as mixed cellulose esters (MCE) membrane, cellulose acetate membrane, cellulose nitrate membrane, PTFE membrane, polycarbonate membrane, track-etched polycarbonate membrane, PCTE (polycarbonate) membrane, polypropylene membrane. PVDF (polyvinylidene difluoride) membrane, or nylon and polyamide membrane.

In one embodiment, the membrane filter is a PET membrane filter with 1 μm pore size. The small pore size can prevent particles larger than 1 μm from passing into the reaction chamber. In another embodiment, the filter assembly may comprise a cotton filter combined with a PET mesh having a pore size of 1 μm.

In some embodiments, the filtration mechanism has low protein binding, low or no nucleic acid binding. The filter may act as a bulk filter to remove fat and emulsifiers and large particles, resulting in a filtrate with viscosity comparable to the viscosity of the buffer.

In some embodiments, the filter assembly 325 including the gross filter 411, the depth filter 412 and the membrane filter 420 can provide maximal recovery of signaling polynucleotides (SPNs) and other detection agents.

In some embodiments, the filtration mechanism can complete the filtering process in less than 1 minute, preferably in about 30 seconds. In one example, the filtration mechanism may be able to collect the sample within 35 seconds, or 30 seconds, or 25 seconds, or 20 seconds with less than 10 psi pressure. In some embodiments, the pressure may be less than 9 psi, or less than 8 psi, or less than 7 psi, or less than 6 psi, or less than 5 psi.

In some alternative embodiments, the filtration chamber 322 may comprise one or more additional chambers configured to filter the processed sample. As illustrated in FIG. 4B, the filtration chamber 322 may further comprise a separate filter bed chamber 431 wherein a filter assembly 325 (as illustrated in FIG. 4A) is inserted and connected to a collection gutter 432. The collection gutter 432 is configured to collect the filtrate that runs through the filter assembly 325, and the gutter 432 may be directly connected to the flow cell fluidics to flow the filtrate to the reaction chamber 331 for signal detection. Optionally, another collection/concentration chamber 433 may be included in the filtration chamber 322 which is configured for collecting and/or concentrating the filtrate collected through the collection gutter 432 before the filtrate is transported to the reaction chamber 331 for signal detection. The collection/concentration chamber 433 is collected to the filter bed chamber 431 through the collection gutter 432.

FIGS. 5A to 5C illustrates an alternative embodiment of the disposable cartridge 300 (FIG. 5A). Similarly, as illustrated in FIG. 5B, the cup comprises three parts, a cup top cover 310, a cup tank 320, and a cup bottom cover 330, which are operatively connected to form an analytic module. The top of the cup is a top cover 310 where a test sample is placed into the cup for testing. A top gasket 501 may be included to seal the top 310 to the cup body. The upper cup body 510 comprises the homogenization chamber, waste chamber, chambers for washing (e.g., wash 1 chamber (W1), wash 2 chamber (W2) which are shown in FIG. 6A), and air vent stacks for controlling air and thus fluid flow. A rotor 340 is configured in the homogenization chamber for homogenizing the test sample in homogenization buffer. The shape of the rotor may be adjusted to fit the cup during the assembly. A mid gasket 502 is located at the bottom of the upper cup body 510 to seal the body 510 to the manifold 520 with holes for fluid flow. The manifold 520 is configured to hold the filter 325 and the fluid paths 370 for fluid flow. Another mid gasket 503 is added to seal the manifold 520 to the bottom cover 330, where the reaction chamber, glass chip, glass gasket and the memory chip (e.g. EPROM) are located. The rotor 340 is sealed to the bottom through an O-ring 505 (shown in FIG. 5C). The rotary valve 350 is configured to the bottom 330 through a valve gasket 504. The configurations of each of the components of the cup shown FIG. 5B are also illustrated in the cross-sectional view of FIG. 5C.

According to the present invention, another alternative embodiment of the disposable cup 300 is illustrated in FIG. 5D. FIGS. 5E to 5K further illustrate the components of the disposable cup 300 of FIG. 5D. As shown in FIG. 5D, the cartridge comprises a top part 310, a body part 320 and a bottom part 330. The rotor 340 is sealed to the cup body through a gasket 533. The rotary valve 350 is assembled to the cartridge through a disc spring 535. When implementing a detection assay, the rotary valve 350 may rotate and move the seal 533 to free the rotor 340 for homogenizing the test sample. In this embodiment, a separate fluidic panel 532 is provided between the bottom of the cup body 320 and the bottom cover 337 in which the fluidic channels are included. When the parts of the test cup are assembled together, the reaction chamber 331 is formed between the fluidic panel 532 and the bottom cover 337. The DNA chip 333 may be operatively connected to the fluidic panel 532 and the sensor area 332 of the reaction chamber 331 through the chip PSA 534. The fluidic paths of the panel 532 will guide the processed sample to the reaction chamber 331 for signal detection.

The cup top 310 may comprise a top lid 311 having two labels 311a and 311b as shown in FIG. 3I. The cup body 320 may be configured to provide several separate chambers, including a homogenization chamber 321, a filtration chamber 322, a waste chamber 323, two or more washing spaces (W1 and W2) as shown in FIG. 5E (upper panel). In some examples, the filtration chamber 322 has a vent 531. The wetting of the vent 531 can signal to the pressure sensor of the electronics that the chamber 322 is full (FIG. 5D). Similar to other designs, at the bottom of the cup body 320, several ports are designed including a port for the rotor 340 and a port for the rotary valve 350 (e.g., the rotary valve 350 shown in FIG. 5I) for assembling a functional cartridge. When the cup bottom cover 337 is sealed to the cup body 320 and seals the cup, these ports are aligned with the ports of the bottom cover 337 (e.g., 340a and 350a as shown in FIG. 5).

In this embodiment, the fluidic panel 532 is inserted to the bottom of the cup body 320; the panel is configured for holding the DNA chip 333 through the chip PSA 534 and provides essential fluid paths (e.g., 370) for flowing the processed sample to the DNA chip 333. FIG. 5K illustrates an exemplary configuration of the fluidic panel 532, wherein the DNA chip 333 may be attached the reaction chamber 331 and the inlet and outlet channels 336 will flow the sample to the DNA chip for detection reaction.

In some examples, a filter assembly 325 is inserted to the homogenization chamber 321 to filtrate the processed sample. In one example, the filter assembly 325 may be the filter illustrated in FIG. 4A. In another example, an alternative filter assembly 525 may be configured to comprise a filter 544 (e.g., a mesh filter) that is inserted to a filter gasket 543, a bulk filter 542 and a filter cap 541 (FIG. 5G). The filter assembly 525 may be fastened by the rotary valve 350 and controlled the valve 350 (FIG. 5H).

In some embodiments, the reaction chamber 331 may comprise a specialized sensing area 332 which is configured for holding a detection sensor for signal detection. In some aspects of the invention, the detection sensor may be a solid substrate (e.g., a glass surface, a chip, and a microwell) of which the surface is coated with capture probes such as short nucleic acid sequences complementary to the SPNs that bind to the target allergen. In some embodiments, the sensing area 332 within the reaction chamber 331 may be a glass chip 333 (FIGS. 3C and 5D).

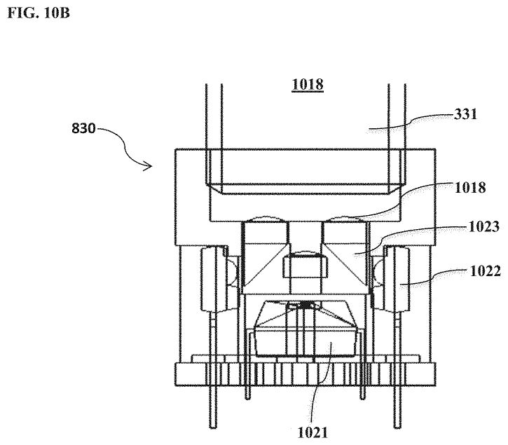

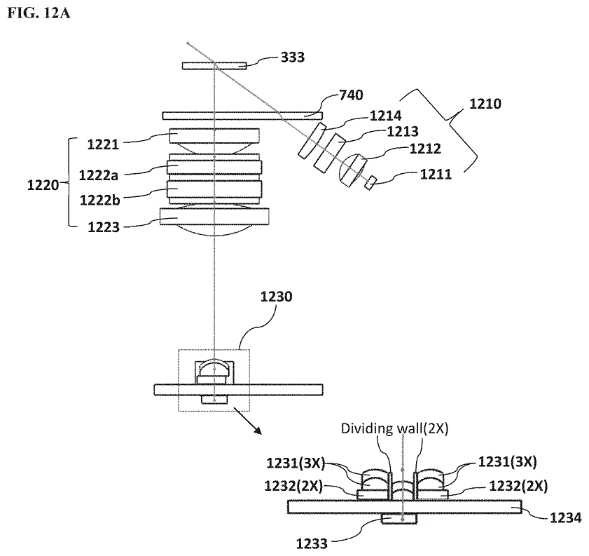

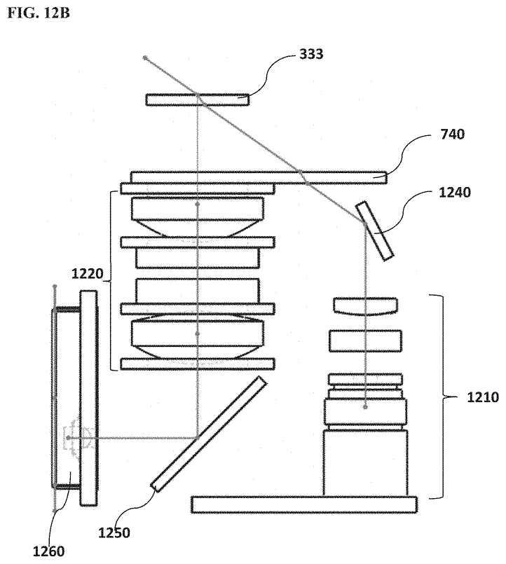

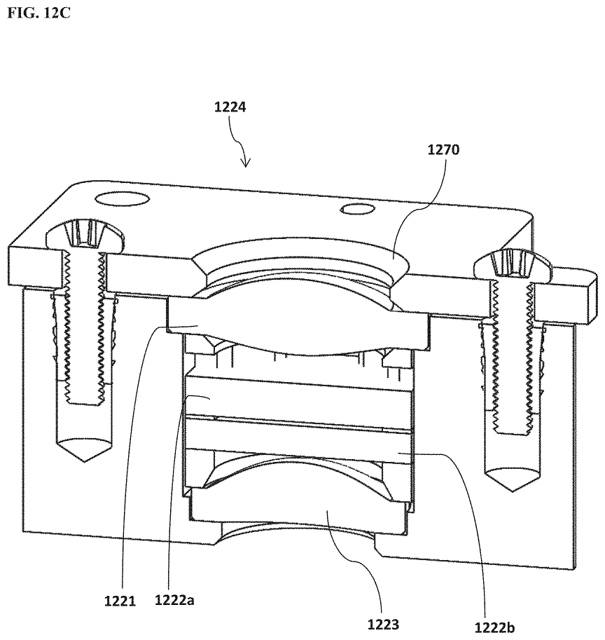

In some embodiments, the reaction chamber 331 comprises at least one optical window. In one embodiment, the chamber comprises two optical windows, one primary optical window and one secondary optical window. In some embodiments, the primary optical window serves as the interface of the reaction chamber 331 to the detection device 100, in particular to the optical system 830 (as shown in FIGS. 10A, 10B, and 12A-12C) of the detection device 100. The detection sensor (e.g., the glass chip 333) may be positioned between the optical window and the interface of the optical system. The optional secondary optical window may be located at one side of the reaction chamber 331. The secondary optical window allows detection of the background signals. In some aspects of the present invention, the secondary optical window may be constructed for measuring scattered light.

In some embodiments, the glass chip 333 printed with nucleic acid molecules (i.e., a DNA chip) is aligned with the optical window. In some embodiments, the DNA chip comprises at least one reaction panel and at least two control panels. In some aspects of the invention, the reaction panel of the chip faces the reaction chamber 331, which is flanked by an inlet and outlet channel 336 of the cartridge 300. In some embodiments, the reaction panel of the glass chip 333 may be coated/printed with short nucleic acid probes that hybridize to a SPN having high specificity and binding affinity to an allergen of interest. The SPN then can be anchored to the chip upon hybridization with the nucleic acid probes.

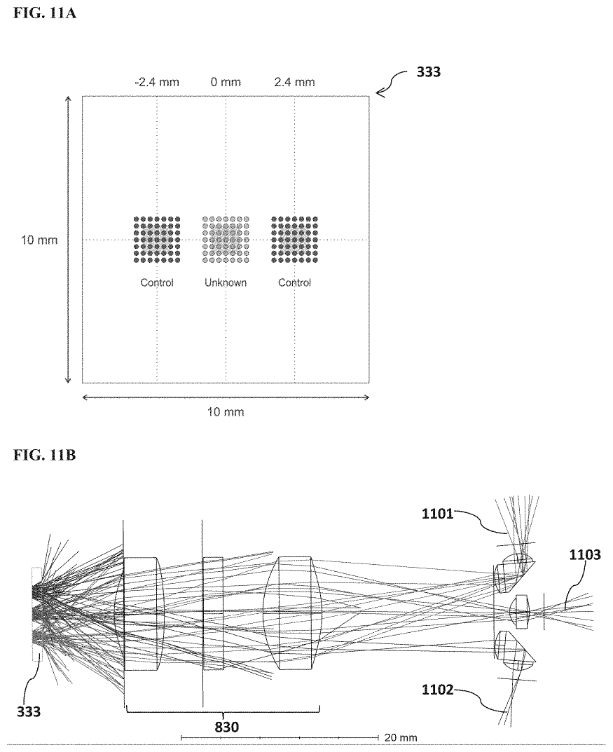

In one preferred embodiment, the sensor DNA chip (e.g., 333 in FIG. 3C) may comprise a reaction panel printed with short complementary sequences that hybridize to a SPN specific to an allergen of interest, and two or more control areas (control panels) that are covalently linked to nucleic acid molecules (as control nucleic acid molecules) that do not react with the SPN or the allergen. The complementary probe sequences can only bind to the SPN when the SPN is free from binding of the target allergen proteins. In some aspects of the invention, the nucleic acid molecules printed in the control panels are labeled with a probe, for example, a fluorophore. The control panels provide an optical set-up with a mechanism to normalize signal output with respect to the reaction panel and to confirm functioning operational procedures. An exemplary configuration of the chip 333 is illustrated in FIG. 11A.

In some embodiments, the DNA coated chip 333 may be pre-packed into the reaction chamber 331 of the cartridge. In other embodiments, the DNA coated chip 333 may be packed separately with the disposable cartridge (e.g. the cup 300 in FIG. 1).

In some embodiments, the solid substrate for making the sensor chip may be a glass with a high optical clarity such as borosilicate glass and soda glass.

In some embodiments, the solid substrate for printing DNA may be made of plastic materials high optical clarity. As non-limiting example, the substrate may be selected from the group consisting of polydimethylsiloxane (PDMS), cyclo-olefin copolymer (COC), polymethylmetharcylate (PMMA), polycarbonate (PC), cyclo-olefin polymer (COP), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polyvinylalcohol, polyacylate, polybutyleneterephthalate (PBT), fluorinated ethylenepropylene (FEP), perfluoralkoxyalkane (PFA), polypropylene carbonate (PPC), polyether sulfone (PES), polyethylene terephthalate (PET), cellulose, poly(4-vinylbenzyl chloride) (PVBC), Toyopearl®, hydrogels, polyimide (PI), 1,2-polybutadiene (PB), fluoropolymers-and copolymers (e.g. poly(tetrafluoroethylene) (PTFE), perfluoroethylene propylene copolymer (FEP), ethylene tetrafluoroethylene (ETFE)), polymers containing norbomene moieties, polymethylmethacrylate, acrylic polymers or copolymers, polystyrene, substituted polystyrene, polyimide, silicone elastomers, fluoropolymers, polyolefins, epoxies, polyurethanes, polyesters, polyethylene terephthalate, polypersulfone, and polyether ketones, or combinations thereof.

The cup bottom 330 is configured to close the disposable test cup 300 and to provide a means for coupling the test cup 300 to the detection device 100. In some embodiments, the bottom side of the bottom assembly 330 of the cup 300 shown in FIG. 3G, includes several interfaces for connecting the cup 300 to the detection device 100 for operation, including a homogenization rotor interface 340a that may couple the homogenization rotor 340 to a motor in the device 100 for controlling homogenization; a valve interface 350a that may couple the rotary valve 350 to a motor in the device 100 for controlling valve rotation; and a pump interface 380 for connecting to a pump in the detection device 100.

In some embodiments, a valve system is provided to control the fluid flow of the sample, detection agents, buffers and other reagents through different parts of the cartridge. In addition to flexible membranes, foil seals and pinch valves discussed herein, other valves may be included to control the flow of the fluid during the process of a detection assay, including swing check valves, gate valves, ball valves, globe valves, rotary valves, custom valves, or other commercially available valves. For example, a gland seal or rotary valve 350 may be used to control the flow of the processed sample solution within the cup 300. In some examples, pinch valves or rotary valves are used to completely isolate the fluid from other internal valve parts. In other examples, air operated valves (e.g., air operated pinch valves) may be used to control the fluid flow, which are operated by a pressurized air supply.

In one embodiment, means for controlling the fluid flow within the cup chambers may be included in, for example, the cup bottom assembly 330. The means may comprise flow channels, tunnels, valves, gaskets, vents and air connections. In one embodiment, the fluidic channels may be configured to the fluidic panel 532 shown in FIG. 5D.

In other embodiments, the valve system of the present invention may comprise additional air vents included in the test cup 300, to control air flow when the DNA coated glass chip is used as the detection sensor. The DNA chip may be purged by air during the course of an allergen detection assay. Individual air intakes may be opened based on the requirements of the system. The valve system discussed herein may be used to keep the air vent unit inactive until use. The air port(s) allow air into the cartridge (e.g., the cup 300) and the air vent(s) allow air to enter various chambers when fluids are added to the chambers or removed from the chambers. The air vent(s) may also have a membrane incorporated therein to prevent spillage and to act as a mechanism to control fluid fill volumes by occlusion of the vent membrane to prevent further flow and fill functions.

In one preferred embodiment, the rotary valve 350 (shown in FIGS. 3C and 5B) may be used to control and regulate fluid flow and rate in the test cup 300. The rotary valve 350 may comprise a valve shaft and a valve disc that can be operated by an associated detection device (e.g., the device 100). In some embodiments, the rotary valve 350 may be positioned at a particular angle by rotating the valve components either counterclockwise (CCW) or clockwise (CW) at each step of the repeated washing and air purge cycle(s) during the process of a detection assay. The air hole permits entry of air. Air is drawn through the system via vacuum pressure to perform air purge functions. The angle may range from about 2° to about 75°.

As a non-limiting example, the valve may be positioned at about 38.5° with respect to the air hole wherein the pump 840 is off and the reaction chamber 331 is dry (referred to as home position). After the test sample is processed and homogenized, the pump is on and the valve 350 is rotated CCW and parks at an angle of about 68.5°, allowing the processed sample to be transported to the filtration chamber 322. Next, the valve components may be rotated again at different directions to park at different angles such as about 57° to flow wash buffer to the reaction chamber 331, and about 72° to purge the DNA chip with air. After the prewash of the DNA chip, the valve components may be rotated to the home position at about 38.5°. The processed sample solution is pulled through the filter assembly 325. After filtration, the valve components may be rotated and park at an angle of about 2°, allowing the collected filtrate to flow into the reaction chamber 331, wherein the chemical reactions occur. The valve 350 will rotate and park at about 57° to flow wash buffer to the reaction chamber 331, and park at about 72° to purge the DNA chip with air. The wash and air purge steps may be repeated one or more times until an optical measurement indicates a clean background.

In one embodiment, the valve system may be a rotary valve operated as shown in FIG. 6B. In this embodiment, the rotary valve 350 is positioned to control air and fluid flow in the system. The of the rotary valve 350 drives the homogenization in the homogenization chamber 321, filtration and collection of filtrate (F), sample washes (e.g., wash 1 (W1) and wash 2 (W2) and waste collection (FIG. 6A). In step 1 of FIG. 6B, the rotary valve 350 is in a closed position with no connections being made between any of the chambers. In step 2 of FIG. 6B, the rotary valve 350 connects the wash 1 chamber W1 to the reaction chamber 331 to flush the reaction chamber 331 with the wash buffer subsequently being pushed out to the waste chamber 323. In step 3 of FIG. 6B, the rotary valve 350 connects the homogenization chamber 321 to the filtrate chamber F to affect the filtration step. In step 4 of FIG. 6B, the rotary valve 350 connects the filtrate chamber F to the reaction chamber 331 to send the filtrate to the reaction chamber 331 for reaction and analysis. In step 5 of FIG. 6B, the rotary valve 350 connects the wash 2 chamber W2 to the reaction chamber to flush the reaction chamber 331 again.

In some embodiments, extraction buffers may be pre-stored in the homogenization chamber 321, for example in foil sealed reservoirs like the food processing reservoir 601 (FIG. 6C). Alternatively, extraction buffers may be stored separately in a separate buffer reservoir in the cup body 320, a reservoir similar to the wash buffer storage reservoir 602 (in the buffer storage chamber 324 (optional) as shown in FIG. 6C). The extraction buffer after sample homogenization and washing waste may be stored in the separate waste reservoir 603 within the waste chamber 323. The waste chamber 323 has sufficient volume to store a volume greater than the amount of fluid used during the detection assay.

In accordance with the present invention, the homogenization rotor 340 may be constructed to be small enough to fit into a disposable test cup 300, particularly into the homogenization chamber 321, where the homogenizer processes a sample to be tested. Additionally, the homogenization rotor 340 may be optimized to increase the efficacy of sample homogenization and protein extraction. In one embodiment, the homogenization rotor 340 may comprise one or more blades or the equivalent thereof at the proximal end. In some examples, the rotor 340 may comprise one, two, three or more blades. The homogenization rotor 340 is configured to pull the test sample from the food corer 200 into the bottom of the homogenization chamber 321.

Alternatively, the homogenization rotor 340 may further comprise a center rod running through the rotor that connects through the cup body 320 to a second interface bit. The central rod may act as an additional bearing surface or be used to deliver rotary motion to the rotor 340. When the rotor 340 is mounted to the cup body 320 through the port at the cup bottom (e.g., 340a), the blade tips may remain submersed within the extraction buffer during operation. In another alternative embodiment, the homogenization rotor 340 may have an extension to provide a pass through the bottom of the cup, the pass may be used as a second bearing support and/or an additional location for power transmission. In this embodiment, the lower part of the rotor has a taper to fit to a shaft, forming a one-piece rotor. In accordance with the present invention, the depth level of the blades of the homogenization rotor 340, with or without the center rod, is positioned to ensure that the blade tips remain in the fluid during sample processing.

As compared to other homogenizers (e.g., U.S. Pat. No. 6,398,402; incorporated herein by reference in its entirety), the custom blade core of the present invention draws and forces food into the toothed surfaces of the custom cap as the blade spins. The homogenizer rotor may be made of any thermoplastics, including, but not limited to, polyamide (PA), acrylanitrilebutadienestyrene (ABS), polycarbonate (PC), high impact polystyrene (HIPS), and acetal (POM).

The disposable cartridge may be in any shape, for example, circular, oval, rectangular, or egg-shaped. Any of these shapes may be provided with a finger cut or notch. The disposable cartridge may be asymmetrical, or symmetrical.

Optionally, a label or a foil seal may be included on the top of the cup lid 311 to provide a final fluid seal and identification of the test cup 300. For example, a designation of peanut indicates that the disposable test cup 300 is used for detecting the peanut allergen in a food sample.

### The Detection Device

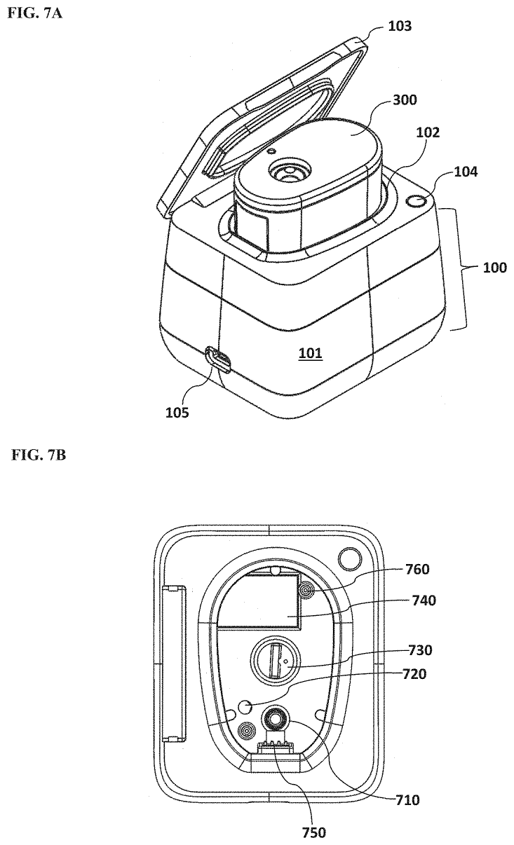

In some embodiments, the detection device 100 may be configured to have an external housing 101 that provides support surfaces for the components of the detection device 100; and a lid 103 that opens the detection device 100 for inserting a disposable test cup 300 and covers the cup during operation. The small lid 103 may be located at one side of the device (as shown in FIG. 1 and FIG. 7A), or in the center (not shown). In some aspects of the invention, the lid may be transparent, allowing all the operations visible through the lid 103. The device may also comprise s USB port 105 for transferring data.

One embodiment of the allergen detection device 100 according to the present invention is depicted in FIG. 1 and FIG. 7A. As illustrated in FIG. 1, the detection device 100 comprising an external housing 101 that provides support for holding the components of the detection device 100 together. The external housing 101 may be formed of plastic or other suitable support material. The device also has a port or receptacle 102 for docking the test cup 300 (FIG. 1 and FIG. 7A).

To execute an allergen detection test, the detection device 100 is provided with a means (e.g., a motor) for operating the homogenization assembly and necessary connectors that connect the motor to the homogenization assembly; means (e.g., a motor) for controlling the rotary valve; means for driving and controlling the flow of the processed sample solution during the process of the allergen detection test; an optical system; means for detecting fluorescence signals from the detection reaction between the allergen in the test sample and the detection agents; means for visualizing the detection signals including converting and digitizing the detected signals; a user interface that displays the test results; and a power supply.

As viewed from the transparent lid 103 (FIG. 7A), the device 100 has an interface comprising areas for coupling the components of the cartridge 300 (when inserted) for operating the reaction (FIG. 7B). These areas include a homogenization bit 710 for coupling the rotor 340 to the motor, a vacuum bit 720 for coupling the cup with the vacuum pump, a rotary valve drive bit 730 for coupling the rotary valve 350 to a valve motor and a protective glass 740 which is aligned to the glass chip 333 through the optical window of the reaction chamber 331. A data chip reader 750 is also included to read the data chip 335. The pins 760 are used to facilitate placement of the cup 300 in the receptacle of the device 100.

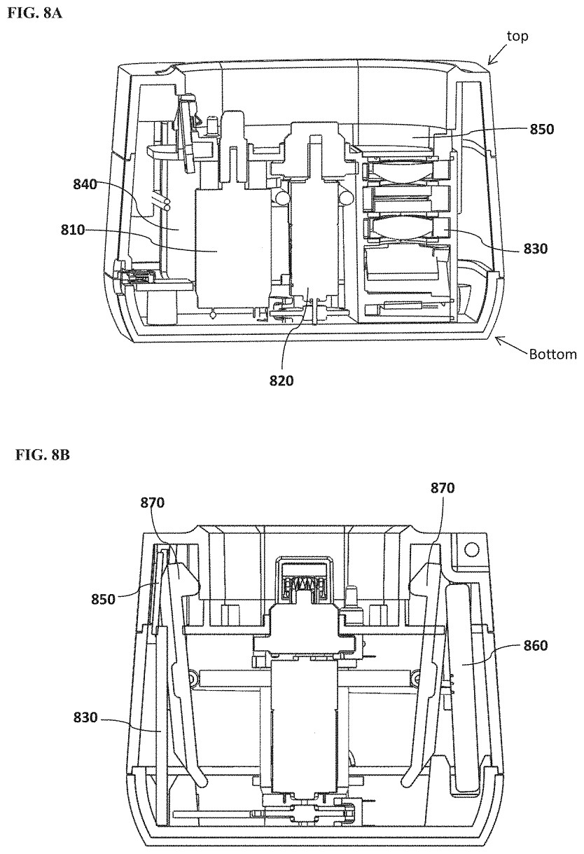

In one embodiment of the present invention, as shown in FIG. 8A, the components of the detection device 100 that are integrated to provide all motion and actuation for operating a detection test, include a motor 810 which may be connected to the homogenization rotor 340 inside the homogenization chamber 321 within the cup body 320. The motor 810 may be connected through a multiple-component coupling assembly including a gear train/drive platen for driving the rotor during homogenization in an allergen detection test; a valve motor 820 for driving the rotary valve 350; an optical system 830 that is connected to the reaction chamber 331 (not shown) of the disposable test cup 300; a vacuum pump 840 for controlling and regulating air and fluid flow (not shown in FIG. 8A), a PCB display 850, and a power supply 860 (in FIG. 8B). A means for retaining the test cup (i.e. the cup retention 870) is included for holding the test cup 300. Each part is described below in detail.

### 1. Homogenization Assembly

In one embodiment, the motor 810 may be connected to the homogenization rotor 340 inside the test cup 300 through the multiple-component rotor coupling assembly. The rotor coupling assembly may include a coupling that is directly linked to the distal end cap of the rotor 340, and a gearhead that is part of a gear train or a drive (not shown) for connection to the motor 810. In some embodiments, the coupling may have different sizes at each end, or the same sizes at each end of the coupling. The distal end of the coupling assembly may connect to the rotor 340 through the rotor port 340a at the cup bottom 330. It is also within the scope of the present invention that other alternative means for connecting the motor 810 to the homogenization rotor 340 may be used to form a functional homogenization assembly.

In some embodiments, the motor 810 can be a commercially available motor, for example, Maxon RE-max and/or Maxon A-max (Maxon Motor ag, San Mateo, Calif., USA).

Optionally, a heating system (e.g. resistance heating, or Peltier heaters) may be provided to increase the temperature of homogenization, therefore, to increase the effectiveness of sample dissociation and shorten the processing time. The temperature may be increased to between 60° C. to 95° C., but should remain at or below 95° C. Increased temperature may also facilitate the binding between detection molecules and the allergen being detected. Optionally a fan or Peltier cooler may be provided to bring the temperature down quickly after implementing the test.

The motor 810 drives the homogenization assembly to homogenize the test sample in the extraction buffer and dissociate/extract allergen proteins. The processed sample solution may be pumped or pressed through the flow tubes to the next chamber for analysis, for example, to the reaction chamber 331 in which the processed sample solution is mixed with the pre-loaded detection molecules (e.g., signaling polynucleotides) for the detection test. Alternatively, the processed sample solution may first be pumped or pressed through the flow tubes to the filter assembly 325 and then to the filtrate chamber 322 before being transported to the reaction chamber 331 for analysis.

### 2. Filtration

In some embodiments, means for controlling the filtration of the processed test sample may be included in the detection device. The food sample will be pressed through a filter membrane or a filtering assembly before the extraction solution being delivered to the reaction chamber 331, and/or other chambers for further processing such as washing. One example is the filter membrane(s). The membranes provide filtration of specific particles from the processed protein solution. For example, the filter membrane may filter particles from about 0.1 μm to about 1000 μm, or about 1 μm to about 600 μm, or about 1 μm to about 100 μm, or about 1 μm to about 20 μm. In some examples, the filter membrane may remove particles up to about 20 μm, or about 19 μm, or about 18 μm, or about 17 μm, or about 16 μm, or about 15 μm, or about 14 μm, or about 13 μm, or about 12 μm, or about 11 μm, or about 10 μm, or about 9 μm, or about 8 μm, or about 7 μm, or about 6 μm, or about 5 μm, or about 4 μm, or about 3 μm, or about 2 μm, or about 1 μm, or about 0.5 μm, or about 0.1 μm. In one example, the filter membrane may remove particles up to about 1 μm from the processes sample. In some aspects, filter membranes may be used in combination to filter specific particles from the assay for analysis. This filter membrane may include multistage filters. Filter membranes and/or filter assemblies may be in any configuration relative to the flow valve. For example, the flow valves may be above, below or in between any of the stages of the filtration.

In some embodiments, the filter assembly may be a complex filter assembly 325 as illustrated in FIG. 4A in which the processed sample is filtered sequentially through the gross filter 411, the depth filter 412 and the membrane filter 420.

### 3. Pump and Fluid Motion

In accordance with the present invention, means for driving and controlling the flow of the processed sample solution is provided. In some embodiments, the means may be a vacuum system or an external pressure. As a non-limiting example, the means may be a platen (e.g., a welded plastic clamshell) configured to being multifunctional in that it could support the axis of the gear train and it could provide the pumping (sealed air channel) for the vacuum to be applied from the pump 840 to the test cup 300. The pump 840 may be connected to the test cup 300 through the pump port 720 located at the bottom (FIG. 7B), which connects to the pump interface 380 (FIG. 3G) on the bottom 330 of the test cup 300 when the cup is inserted to the device.

The pump 840, may be a piezoelectric micro pump (e.g., Takasago Electric, Inc., Nagoya. Japan) or a peristaltic pump, which may be used to control and automatically adjust the flow to a target flow rate. The flow rate of a pump is adjustable by changing either the driver voltage or drive frequency. As a non-limiting example, the pump 840 may be a peristaltic pump. In another embodiment, the pump 840 may be a piezoelectric pump currently on the market that has specifications suitable for the aliquot function required to flow filtered sample solution to different chambers inside the test cup 300. The pump 840 may be a vacuum pump or another small pump constructed for laboratory use such as a KBF pump (e.g., KNF Neuberger, Trenton, N.J., USA).

Alternatively, a syringe pump, diaphragm and/or a micro-peristaltic pump may be used to control fluid motion during the process of a detection assay and/or operation of supporting fluidics. In one example, an air operated diaphragm pump may be used.

### 4. Rotary Valve Control

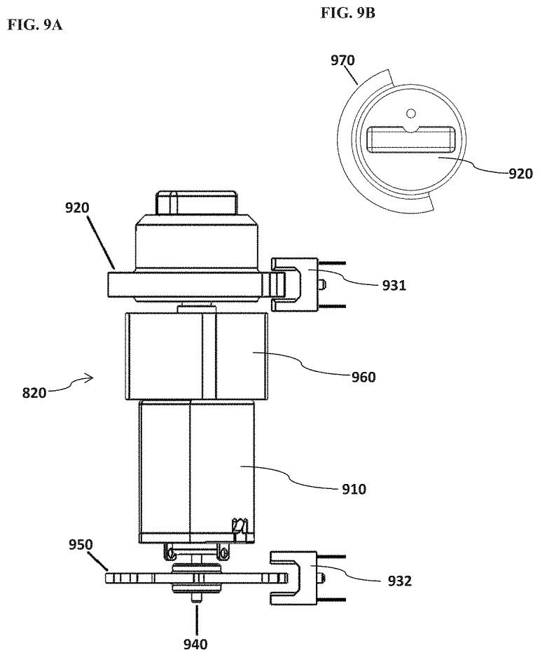

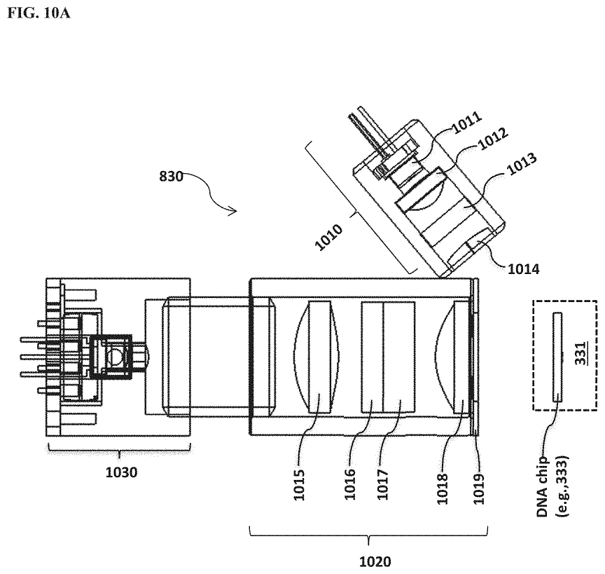

In some embodiments, the rotary valve 350 (e.g., as shown in FIG. 5I) for controlling fluid flow needs to be in precise positions. A means to control the rotary valve is provided and the control mechanism is able to rotate the valve in both directions and accurately stop at desired locations. In some embodiments, the device 100 includes a valve motor 820 (in FIG. 7B). As shown in FIG. 9A, the valve motor 820 may be a low cost, DC geared motor 910 with two low cost optical sensors (931 and 932), and a microcontroller. An output coupling 920 interfaces with the rotary valve 350. In some embodiments, the output coupling 920 has a shelf 970 with a half-moon shape as shown in FIG. 9B, which interrupts the output optical sensor 931 with the protruding half. The output optical sensor signal toggles between high and low, depending on whether or not the protruding shelf interrupts the sensor. A microcontroller (MCU) detects these transitions and obtains an absolute position of the output from this signal. The positioning of these transitions is important and application-specific since these transitions are used during directional changes to account for gear backlash.

The direct motor shaft 940 has a paddle wheel which interrupts the direct shaft optical sensor 932, allowing the direct shaft optical sensor 932 to output a train of pulses, with the number of pulses per revolution determined by the number of paddles on the wheel 950. The MCU reads this train of pulses and determines the degrees movement of the output coupling. The resolution is dependent on the number of paddles of the direct shaft encoder wheel 950, and the gear reduction ratio of the gear box 960.

The MCU interprets the output of these two optical sensors and can drive the output to a desired location, as long as the position of the output coupling shelf transitions, the number of paddle wheels on the direct encoder wheel 920, and the gear ratio are known. During a change of direction, the motor must rotate by a fixed amount before an output transition is seen. The fixed amount is selected to overcome backlash of the gears. Once the fixed amount is overcome, on the next output signal transition, the MCU can start counting the direct signal pulses with confidence that they correspond to accurate output of location and movement.

### 5. Optical System

The detection device 100 of the present invention comprises an optical system that detects optical signals (e.g., a fluorescence signal) generated from the interaction between an allergen in the sample and detection agents (e.g., aptamers and SPNs). The optical system may comprise different components and variable configurations depending on the types of the fluorescence signal to be detected. The optical system is close to and aligned with the detection cartridge, for instance, the primary optical window and optionally the secondary optical window of the reaction chamber 331 of the test cup 300 as discussed above.

In some embodiments, the optical system 830 may include excitation optics 1010 and emission optics 1020 (FIGS. 10A and 10B). In one embodiment, as shown in FIG. 10A, the excitation optics 1010 may comprise a laser diode 1011 configured to transmit an excitation optical signal to the sensing area (e.g., 332) in the reaction chamber 331, a collimation lens 1012 configured to focus the light from the light source, a filter 1013 (e.g., a bandpass filter), a focus lens 1014, and an optional LED power monitoring photodiode. The emission optics 1020 may comprise a focus lens 1015 configured to focus at least one portion of the allergen-dependent optical signal onto the detector (photodiode), two filters including a longpass filter 1016 and a bandpass filter 1017, a collection lens 1018 configured to collect light emitted from the reaction chamber and an aperture 1019. The emission optics collects light emitted from the solid surface (e.g. a DNA chip) in the detection chamber 331 and the signal is detected by the detector 1030 configured to detect an allergen-dependent optical signal emitted from the sensing area 332. In some aspects, the excitation power monitoring may be integrated into the laser diode 1011 (not shown in FIG. 10A).

A light source 1011 is arranged to transmit excitation light within the excitation wavelength range. Suitable light sources include, without limitation, lasers, semi-conductor lasers, light emitting diodes (LEDs), and organic LEDs.

An optical lens 1012 may be used along with the light source 1011 to provide excitation source light to the fluorophore. An optical lens 1014 may be used to limit the range of excitation light wavelengths. In some aspects, the filter may be a bandpass filter.

Fluorophore labeled SPNs specific to a target allergen are capable of emitting, in response to excitation light in at least one excitation wavelength range, an allergen-binding dependent optical signal (e.g. fluorescence) in at least one emission wavelength range.

In some embodiments, the emission optics 1020 are operable to collect emissions upon the interaction between detection agents and target allergens in the test sample from the reaction chamber 331. Optionally, a mirror may be inserted between the emission optics 1020 and the detector 1030. The mirror can rotate in a wide range of angles (e.g., from 1° to 90°) which could facilitate formation of a compacted optical unit inside the small portable detection device.

In some embodiments, more than one emission optical system 1020 may be included in the detection device. As a non-limiting example, three photodiode optical systems may be provided to measure fluorescence signals from an unknown test area and two control areas on a glass chip (e.g., see FIG. 11B). In other aspects, an additional collection lens 1018 may be further included in the emission optics 1020. This collection lens may be configured to detect several different signals from the chip 333. For example, when the detection assay is implemented using a DNA glass chip, more than two control areas may be constructed on the solid surface in addition to a detection area for allergen detection. The internal control signals from each control area may be detected at the same time when an allergen-derived signal is measured. In this context, more than two collection lenses 1018 may be included in the optical system 830, one lens 1018 for signal from the detection area and the remaining collection lenses 1018 for signals from the control areas.

The detector (e.g., photodiode) 1030 is arranged to detect light emitted from the fluidic chip in the emission wavelength range. Suitable detectors include, without limitation, photodiodes, complementary metal-oxide-semiconductor (CMOS) detectors, photomultiplier tubes (PMT), microchannel plate detectors, quantum dot photoconductors, phototransistors, photoresistors, active-pixel sensors (APSs), gaseous ionization detectors, or charge-coupled device (CCD) detectors. In some aspects, a single and/or universal detector can be used.

In some embodiments, the optical system 830 may be configured to detect fluorescence signals from the solid substrate (e.g., DNA chip 333 shown in FIG. 11A). The DNA chip may be configured to contain a central reaction panel which is marked as an “unknown” signal area on the chip (FIG. 11A), and at least two control areas at various locations of the chip (FIG. 11A). In this context, the optical system 830 is configured to measure both detection signals and internal control signals simultaneously (FIG. 11B).

In one example, the optical system 830 comprises two collection lenses 1018 and corresponding optical components, such as control array photodiodes for each lens 1018. FIG. 10B demonstrates a side view of the optical system 830 shown in FIG. 10A inside the detection device 100. In this embodiment, two collection lenses 1018 are included in the optical system, one for collecting control array signals from the DNA chip (e.g., the two signals 1101 and 1102 shown in FIG. 11B) and one specific to the unknown detection signal from the DNA chip (e.g., the detection signal 1102 as shown in FIG. 11B). A signal array diode 1021 (e.g., the laser diode 1011 shown in FIG. 10A) and two control assay photodiodes 1022 are included for each optical path. Additionally, two prisms 1023 may be added to the two collection-lenses (1018) configured for collecting signals from the two control areas. The prisms 1023 can bend the control array light to the photodiode sensor area.

In some embodiments, the optical system 830 may be configured as a straight mode as shown in FIG. 12A. The excitation optics 1210, which are configured to transmit an excitation optical signal to the glass chip 333 (e.g., DNA coated chip) in the reaction chamber 331, may comprise a laser diode 1211, a collimation lens 1212, a bandpass filter 1213 and a cylinder lens 1214. The cylinder lens 1214 may cause the excitation light to form a line to cover the reaction panel and control panels on the glass chip (e.g., FIG. 11B). The emission optics 1220 which are aligned with the glass chip 333 may comprise a collection lens 1221 configured to collect light emitted from the glass chip 333, a bandpass filter 1222a, a longpass filter 1222b, and a focus lens 1223 configured to focus at least one portion of the allergen-dependent optical signal onto the chip reader 1230. The chip reader 1230 is composed of three photodiode lenses 1231, two control array photodiodes 1232, a signal array photodiode 1233 and a collection PCB 1234 (FIG. 12A). In some embodiments, the collection lens 1221 may be shaped to contain a concave first surface to optimize imaging and minimize stray light.

As a non-limiting example, the excitation optics 1210 and the emission optics 1220 may be folded and configured into a stepped bore 1224 in the device 100 (see FIG. 12C). An excitation folding mirror 1240 and a collection folding mirror 1250 may be configured to minimize the light paths from the excitation optics 1210 and the emission optics 1220, respectively (in FIG. 12B). The minimized volume can modulate the laser at a frequency to minimize interference from environmental light sources. A photodiode shield 1260 may be added to cover and protect the chip reader 1230 (FIG. 12B). The reader 1230 is then positioned close to the collection lens 1221 to minimize the scattered light. FIG. 12C illustrates an example of the stepped bore 1224 in the device to hold the emission optics 1220. The aperture 1270 of the collection lens 1221 is shown in FIG. 12C.

The laser source (e.g., 1211) may be modulated, and/or polarized and oriented to minimize the reflections from the glass chip. Accordingly, the chip reader may be synchronized to measure modulated light.

The above described optical system 830 is illustrative examples of certain embodiments. Alternative embodiments might have different configurations and/or different components.

In other embodiments, a computer or other digital control system can be used to communicate with the light filters, the fluorescence detector, the absorption detector and the scattered detector. The computer or other digital control systems control the light filter to subsequently illuminate the sample with each of the plurality of wavelengths while measuring absorption and fluorescence of the sample based on signals received from the fluorescence and absorption detectors.

### 6. Display

As shown in a cut-away side view in FIG. 8B, a printed circuit board (PCB) 850 is connected to the optical system 830. The PCB 850 may be configured to be compact with the size of the detection device 100 and at the same time, may provide enough space to display the test result.

Accordingly, the test result may be displayed with back lit icons, LEDs or an LCD screen, OLED, segmented display or on an attached mobile phone application. The user may see an indicator that the sample is being processed, that the sample was processed completely (total protein indictor) and the results of the test. The user may also be able to view the status of the battery and what kind of cartridge is placed in the device (bar code on the cartridge or LED assembly). The results of the test will be displayed, for example, as (1) actual number ppm or mg; or (2) binary result yes/no; or (3) risk analysis—high/medium/low or high/low, risk of presence; or (4) range of ppm less than 1/1-10 ppm/more than 10 ppm; or (5) range of mg less than 1 mg/between 1-10 mg/more than 10 mg. The result might also be displayed as numbers, colors, icons and/or letters.

In accordance with the present invention, the detection device 100 may also include other features such as means for providing a power supply and means for providing control of the process. In some embodiments, one or more switches are provided to connect the motor, the micropump and/or the gear train or the drive to the power supply. The switches may be simple microswitches that can turn the detection device on and off by connecting and disconnecting the battery.

The power supply 860 may be a Li-ion AA format battery or any commercially available batteries that are suitable for supporting small medical devices such as the Rhino 610 battery, the Turntigy Nanotech High dischargeable Li Po battery, or the Pentax D-L163 battery.

In the description herein, it is understood that all recited connections between components can be direct operative connections or indirectly operative connections. Other components may also include those disclosed in the applicant's PCT patent publication NO. WO/2018/156535; the contents of which are incorporated herein by reference in their entirety.

### Detection Assays

In another aspect of the present invention, there is provided an allergen detection test implemented using the present detection systems and devices.

In some embodiments, the allergen detection test comprises the steps of (a) collecting a certain amount of a test sample suspected of containing an allergen of interest, (b) homogenizing the sample and extracting allergen proteins using an extraction/homogenization buffer, (c) contacting the processed sample with a detection agent that specifically binds to a target allergen; (d) contacting the mixture in (c) with a detection sensor comprising a solid substrate that is printed with nucleic acid probes; (e) measuring fluorescence signals from the reaction; and (f) processing and digitizing the detected signals and visualizing the interaction between the detection agents and the allergen.

In some aspects of the invention, the method further comprises the step of washing off the unbound compounds from the detection sensor to remove any non-specific binding interactions.

In some aspects of the invention, the method further comprises the step of filtering of the processed sample prior to contacting it with the detection sensor (e.g., DNA chip).

In some embodiments, an appropriately sized test sample is collected for the detection assay to provide a reliable and sensitive result from the assay. In some examples, a sampling mechanism that can collect a test sample effectively and non-destructively for fast and efficient extraction of allergen proteins for detection is used.

A sized portion of the test sample can be collected using, for example, a food corer 200 illustrated in FIG. 2B. The food corer 200 can collect an appropriately sized sample from which sufficient protein can be extracted for the detection test. The sized portion may range in mass from 0.1 g to 1 g, preferably 0.5 g. Furthermore, the food corer 200 may pre-process the collected test sample by cutting, grinding, blending, abrading and/or filtering. Pre-processed test sample will be introduced into the homogenization chamber 321 for processing and allergen protein extraction.

The collected test sample is processed in an extraction/homogenization buffer. In some aspects, the extraction buffer is stored in the homogenization chamber 321 and may be mixed with the test sample by the homogenization rotor 340. In other aspects, the extraction buffer may be released into the homogenization chamber 321 from another separate storage chamber. The test sample and the extraction buffer will be mixed together by the homogenization rotor 340 and the sample being homogenized.

The extraction buffer may be universal target extraction buffer that can retrieve enough target proteins from any test sample and be optimized for maximizing protein extraction. In some embodiments, the formulation of the universal protein extraction buffer can extract the protein at room temperature and in minimal time (less than 1 min). The same buffer may be used during food sampling, homogenization and filtering. The extraction buffer may be PBS based buffer containing 10%, 20% or 40% ethanol, or Tris based buffer containing Tris base pH 8.0, 5 mM MEDTA and 20% ethanol, or a modified PBS or Tris buffer. In some examples, the buffer may be a HEPES based buffer. Some examples of modified PBS buffers may include: P+ buffer and K buffer. Some examples of Tris based buffers may include Buffer A+, Buffer A, B, C, D, E, and Buffer T. In some embodiments, the extraction buffer may be optimized for increasing protein extraction. A detailed description of each modified buffer is disclosed in the PCT Patent Publication No.: WO/2015/066027: the content of which is incorporated herein by reference in its entirety.

In accordance with the present invention, MgCl₂ is added after the sample is homogenized. In some embodiments, MgCl₂ solution (e.g., 30 μL of 1 M MgCl₂ solution) is added to the homogenization chamber (e.g., 321 in FIG. 3) after the sample homogenization.

In other embodiments, solid MgCl₂ formulations may be used instead of MgCl₂ solution during the reaction. The solid formulation may be provided as a MgCl₂ lyophilized pellet in the homogenization chamber (e.g., 321 in FIG. 3) which is dissolved by the homogenate after filtration, or a filter component deposited or layered in the filter (e.g., the filter membrane 420 in FIG. 4A and the filter assembly 325 in FIG. 4A, or the filter assembly 525 in FIG. 5G) that is dissolved by the homogenate during the filtration, or a MgCl₂ film deposited on the inner surface of the homogenization chamber 321), or on a separate support. Regardless of the formulations, MgCl₂ will dissolve in less than 1 minute, preferably in less than 30 seconds, to be contacted with the processed sample homogenate. MgCl₂ may dissolve in about 10 seconds, or about 15 seconds, or about 20 seconds, or about 25 seconds, or about 30 seconds. The solid formulation will release MgCl₂ within this short period of time to reach to a final concentration of 30 mM. In some aspects, the solid MgCl₂ formulation may not break up into powder.

The volume of the extraction buffer may be from 0.5 mL to 3.0 mL. In some embodiments, the volume of the extraction buffer may be 0.5 mL, 1.0 mL, 1.5 mL, 2.0 mL, 2.5 mL or 3.0 mL. The volume has been determined to be efficient and repeatable over time and in different food matrices.

In accordance with the present invention, the test sample is homogenized and processed using the homogenization assembly that has been optimized with high speed homogenization for maximally processing the test sample.

In some aspects of the invention, a filtering mechanism may be linked to the homogenizer. The homogenized sample solution is then driven to flow through a filter in a process to further extract allergen proteins and remove particles that may interfere with the flow and optical measurements during the test, lowering the amounts of other molecules extracted from the test sample. The filtration step may further achieve uniform viscosity of the sample to control fluidics during the assay. In the context that DNA glass chips are used as detection sensors, the filtration may remove fats and emulsifiers that may adhere to the chip and interfere with the optical measurements during the test. In some embodiments, a filter membrane such as a cell strainer from CORNING (CORNING, NY, USA) or similar custom embodiment may be connected to the homogenizer. The filtering process may be a multi-stage arrangement with different pore sizes from the first filter to the second filter, or to the third filter. The filtering process may be adjusted and optimized depending on food matrices being tested. As a non-limiting example, a filter assembly with a small pore size may be used to capture particles and to absorb large volumes of liquid when processing dry foods, therefore, longer times and higher pressures may be used during the filtration. In another example, bulk filtration may be implemented to absorb fat and emulsifiers when processing fatty foods. The filtration may further facilitate to remove fluorescence haze or particles from fluorescence foods, which will interfere with the optical measurements.

The filter may be a simple membrane filter, or an assembly composed of a combination of filter materials such as PET, cotton and sand, etc. In some embodiments, the homogenized sample may be filtered through a filter membrane, or a filter assembly. e.g., the filter assembly 325 in FIG. 4A.

In some aspects of the present invention, the sampling procedure may reach effective protein extraction in less than 1 minute. In one aspect, speed of digestion may be less than 2 minutes including food pickup, digestion and readout. Approximately, the procedure may last 15 seconds, 30 seconds, 45 seconds, 50 seconds, 55 seconds, 1 minute or 2 minutes.

Extracted allergen proteins may be mixed with one or more detection agents that are specific to one or more allergens of interest. The interaction between allergen protein extraction and detection agents will generate a detectable signal which indicates the presence, or absence or the amount of one or more allergens in the test sample. As used herein, the term “detection agent” or “allergen detection agent” refers to any molecule which interacts with and/or binds to one or more allergens in a way that allows detection of such allergens in a sample. The detection agent may be a protein-based agent such as an antibody, a nucleic acid-based agent or a small molecule.

In some embodiments, the detection agent is a nucleic acid molecule based signaling polynucleotide (SPN). The SPN comprises a core nucleic acid sequence that binds to a target allergen protein with high specificity and affinity. The core nucleic acid sequence may be 5-100 nucleic acids in length, or 10-80 nucleic acids in length, or 10-50 nucleic acids in length. The SPN may be derived from an aptamer selected by a SELEX method. As used herein, the term “aptamer” refers to a nucleic acid species that has been engineered through repeated rounds of in vitro selection or equivalently, SELEX (systematic evolution of ligands by exponential enrichment) to bind to various molecular targets such as small molecules, proteins, nucleic acids, and even cells, tissues and organisms. The binding specificity and high affinity for target molecules, the sensitivity and reproductively at ambient temperature, the relatively low production cost, and the possibility to develop an aptamer core sequence that can recognize any protein, ensure an effective but simple detection assay.

In accordance with the present invention, SPNs that can be used as detection agents may be aptamers specific to a common allergen such as peanut, tree-nut, fish, gluten, milk and egg. For example, the detection agent may be the aptamers or SPNs described in applicants' relevant U.S. Provisional Application Ser. No. 62/418,984, filed on Nov. 8, 2016, 62/435,106, filed on Dec. 16, 2016, and 62/512,299 filed on May 30, 2017; and PCT Publication No.: WO/2018/089391 filed on Nov. 8, 2017, the contents of which are incorporated herein by reference in their entirety.

In some embodiments, the detection agent (e.g., an SPN) may be labeled with a fluorescence marker. The fluorescence marker may be a fluorophore with a suitable excitation maximum in the range of 200 to 700 nm, while the emission maximum may be in the range of 300 to 800 nm. The fluorophore may further have a fluorescence relaxation time in the range of 1-7 nanoseconds, preferably 3-5 nanoseconds. As non-limiting examples, a fluorophore that can be probed at one terminus of an SPN may include derivatives of boron-dipyrromethene (BODIPY, e.g., BODIPY TMR dye and BODIPY FL dye), fluorescein and derivatives thereof, rhodamine and derivatives thereof, dansyls and derivatives thereof (e.g. dansyl cadaverine), Texas red, eosin, cyanine dyes, indocarbocyanine, oxacarbocyanine, thiacarbocyanine, merocyanine, squaraines and derivatives seta, setau, and square dyes, naphthalene and derivatives thereof, coumarin and derivatives thereof, pyridyloxazole, nitrobenzoxadiazole, benzoxadiazole, anthraquinones, pyrene and derivatives thereof, oxazine and derivatives, Nile red, Nile blue, cresyl violet, oxazine 170, proflavin, acridine orange, acridine yellow, auramine, crystal violet, malachite green, porphin, phthalocyanine, bilirubin, tetramethylrhodamine, hydroxycoumarin, aminocoumarin: methoxycoumarin, cascade blue, pacific blue, pacific orange, NBD, r-phycoerythrin (PE), red 613; perCP, trured; fluorX, Cy2, Cy3, Cy5 and Cy7, TRITC, X-rhodamine, lissamine rhodamine B, allophycocyanin (APC) and Alexa Fluor® dyes (e.g., Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 637, Alexa Fluor® 647, Alexa Fluor® 660, Alexa Fluor-® 680, and Alexa Fluor® 700).

In one example, the SPN is labeled with Cy5 at the 5′ end of the SPN nucleic acid sequence. In another example, the SPN is labeled with Alexa Fluor® 647 at the one end of the SPN nucleic acid sequence.

In some embodiments, the SPN specific to an allergen of interest may be pre-stored in the extraction/homogenization buffer in the homogenization chamber 321 (FIGS. 3B and 3E). The extracted allergen protein, if present in the test sample, will bind to the SPN, forming a protein:SPN complex. This protein:SPN complex can be detected by a detection sensor during the test process.

In some embodiments, detection agents for eight major food allergens (i.e. wheat, egg, milk, peanuts, tree nuts, fish, shell-fish and soy) may be provided as disposables. In one aspect, constructs of the detection agents may be stored with MgCl₂, or buffer doped with KCL. MgCl₂ keeps constructs closed tightly, while KCl opens them slightly for bonding.

In some embodiments, the detection sensor is a nucleic acid printed solid substrate. As used herein, the term “detection sensor” refers to an instrument that can capture a reaction signal, i.e. the reaction signal derived from the binding of allergen proteins and detection agents, measure a quantity and/or a quality of a target, and convert the measurement to a signal that can be measured digitally.

In some embodiments, the detection sensor is a solid substrate, such as a glass chip, coated with nucleic acid molecules (as referred to herein as nucleic acid chip or DNA chip). For example, the detection sensor may be the glass chip 333 inserted into the reaction chamber 331 of the present invention. The detection sensor may also be a separate glass chip, for example, prepared from glass wafer and soda glass, or a microwell, or an acrylic glass, or a microchip, or a plastic chip made of COC (cyclic olefin copolymer) and COP (cyclo-olefin polymer), or a membrane like substrate (e.g., nitrocellulose), of which the surface is coated with nucleic acid molecules.

In some embodiments, the nucleic acid coated chip may comprise at least one reaction panel and at least two control panels. The reaction panel is printed with nucleic acid probes that hybridize to the SPN. As used herein, the term “nucleic acid probe” refers to a short oligonucleotide comprising a nucleic acid sequence complementary to the nucleic acid sequence of an SPN. The short complementary sequence of the probe can hybridize to the free SPN. When the SPN is not bound by a target allergen, the SPN can be anchored to the probe through hybridization. When the SPN binds to a target allergen to form a protein:SPN complex, the protein:SPN complex prevents hybridization between the SPN and its nucleic acid probe.

In some examples, the probe comprises a short nucleic acid sequence that is complementary to the sequence of the 3′ end of the SPN that specifically binds to a target allergen protein. In this context, the SPN specific to the target allergen protein is provided in the extraction/homogenization buffer. When the sample is processed in the homogenization chamber 321, the target allergen, if present in the test sample, will bind to the SPN, and form a protein:SPN complex. When the sample solution flows to the detection sensor, e.g., the DNA chip 333 in the reaction chamber 331 (FIG. 3B), the bound allergen protein prevents the SPN from hybridizing to the complementary SPN probes on the chip surface. The protein:SPN complex is washed off and no fluorescence signal is detected. In the absence of the target allergen proteins in the test sample, the free SPN will bind to the complementary SPN probes on the chip surface. A fluorescence signal will be detected from the reaction panel (as shown in FIGS. 11A and 11B).

In some embodiments, the detection sensor, e.g., nucleic acid printed chip, further comprises at least two control panels. The control panels are printed with nucleic acid molecules that do not bind to an SPN or a protein (referred herein as “control nucleic acid molecules”). In some examples, the control nucleic acid molecules are labeled with a fluorescence marker.

In some embodiments, nucleic acid probes may be printed to a reaction panel at the center of a glass chip (“urknown”) and control nucleic acid molecules may be printed to the two control panels at each side of the reaction panel on the glass chip, as illustrated in FIG. 11A.

In some embodiments, the nucleic acid chip (DNA chip) may be prepared by any known DNA printing technologies known in the art. In some embodiments, the DNA chip may be prepared by using single spot pipetting to pipette nucleic acid solution onto the glass chip, or by stamping with a wet PDMS stamp comprising a nucleic acid probe solution followed by pressing the stamp against the glass slide, or by flow with microfluidic incubation chambers.

As a non-limiting example, a glass wafer can be laser cut to produce 10×10 mm glass “chips”. Each chip contains three panels: one reaction panel (i.e. the “unknown” area in the chip demonstrated in FIG. 11A) that is flanked by two control panels (FIG. 11A). The reaction panel contains covalently-bound short complementary nucleic acid probes to which SPNs specific to an allergen protein bind. The SPNs are derived from aptamers and modified to contain a CY5 fluorophore. In the absence of the target allergen protein, SPNs are free to bind to the probes in the reaction panel, resulting in a high fluorescence signal. In the presence of the target allergen protein, the SPN: probe hybridizing interface is occluded by the binding of the target protein to the SPNs, thereby resulting in a decrease in fluorescence signal on the reaction panel. In a detection assay, the reaction panel of the chip faces a small reaction chamber (e.g. the reaction chamber 331) flanked by an inlet and outlet channel (e.g., 336 in FIG. 3G) of the cartridge (e.g., the cup 300). During food homogenization, the SPN in the extraction buffer binds to the target allergen if it is present in the sample forming a protein:SPN complex. The processed sample solution including the protein:SPN complex enters the reaction chamber 331 via the inlet, through fluidic movement driven by a vacuum pump. The solution then exits into a waste chamber 323 via the outlet channel. After exposure to the sample, the reaction panel is then washed, revealing a fluorescence signal with an intensity correlated to the target allergen concentration.

In accordance with the present invention, the two control panels are constantly bright areas on the chip sensor that produce a constant signal as background signals 1101 and 1102 (FIG. 11B). In addition, the two control panels compensate for laser illumination and/or disposable cartridge misalignment. If the cartridge is perfectly aligned, then the fluorescence background signals 1101 and 1102 would be equal (as shown in FIG. 11B). If the measured control signals are not equal, then a look-up table of correction factors will be used to correct the unknown signal as a function of cartridge/laser misalignment. The final measurement is a comparison of the signal 1103 of the unknown test area against the signal levels of the control areas. The comparison level may be one of the lot-specific parameters for the test.

Food samples with high background fluorescence measurements from the reaction area may produce a false negative result. A verification method may be provided to adjust the process.

The final fluorescence measurement of the reaction panel, after being compared to the controls and any lot specific parameters may be analyzed and a report of the result may be provided.

Accordingly, the light absorption and light scattering signals may also be measured at the baseline level, before and/or after the injection of the processed food sample. These measurements will provide additional parameters to adjust the detection assay. For example, such signals may be used to look for residual food in the reaction chamber 331 after the wash step.

In addition to the parameters discussed above, one or more other lot-specific parameters may also be measured. The optimization of the parameters, for example, may minimize the disparity in the control and unknown signal levels for the chips.

In some embodiments, the monitoring process may be automatic and controlled by a software application. Evaluation of the DNA chip and test sample, the washing process and the final signal measurement may be monitored during the detection assay.

Allergen families that can be detected using the detection system and device described herein include allergens from foods, the environment or from non-human proteins such as domestic pet dander. Food allergens include, but are not limited to proteins in legumes such as peanuts, peas, lentils and beans, as well as the legume-related plant lupin, tree nuts such as almond, cashew, walnut, Brazil nut, filbert/hazelnut, pecan, pistachio, beechnut, butternut, chestnut, chinquapin nut, coconut, ginkgo nut, lychee nut, macadamia nut, nangai nut and pine nut, egg, fish, shellfish such as crab, crawfish, lobster, shrimp and prawns, mollusks such as clams, oysters, mussels and scallops, milk, soy, wheat, gluten, corn, meat such as beef, pork, mutton and chicken, gelatin, sulphite, seeds such as sesame, sunflower and poppy seeds, and spices such as coriander, garlic and mustard, fruits, vegetables such as celery, and rice. The allergen may be present in a flour or meal, or in any format of products. For example, the seeds from plants, such as lupin, sunflower or poppy can be used in foods such as seeded bread or can be ground to make flour to be used in making bread or pastries.

### Applications

The detection systems, devices and methods described herein contemplate the use of nucleic acid-based detector molecules such as aptamers for detection of allergens in food samples. The portable devices allow a user to test the presence or absence of one or more allergens in food samples. Allergen families that can be detected using the device described herein include allergens from legumes such as peanuts, tree nuts, eggs, milk, soy, spices, seeds, fish, shellfish, wheat gluten, rice, fruits and vegetables. The allergen may be present in a flour or meal. The device is capable of confirming the presence or absence of these allergens as well as quantifying the amounts of these allergens.

In a broad concept, the detection systems, devices and methods described herein may be used for detection of any protein content in a sample in a large variety of applications in addition to food safety, such as, for example, medical diagnosis of diseases in civilian and battlefield settings, environmental monitoring/control and military use for the detection of biological weapons. In even broad applications, the detection systems, devices and methods of the present invention may be used to detect any biomolecules to which nucleic acid-based detector molecules bind. As some non-limiting examples, the detection systems, devices and methods may be used for on-the-spot detection of cancer markers, in-field diagnostics (exposure the chemical agents, traumatic head injuries etc.), third-world applications (TB, HIV tests etc.), emergency care (stroke markers, head injury etc.) and many others.

As another non-limiting example, the detection systems, devices and methods of the present invention can detect and identify pathogenic microorganisms in a sample. Pathogens that can be detected include bacteria, yeasts, fungi, viruses and virus-like organisms. Pathogens cause diseases in animals and plants; contaminate food, water, soil or other sources: and are used as biological weapons. The device is capable of detecting and identifying pathogens.

Another important application includes the use of the detection systems, devices and methods of the present invention for medical care, for example, to diagnose a disease, to stage a disease progression and to monitor a response to a certain treatment. As a non-limiting example, the detection device of the present invention may be used to test the presence or absence, or the amount of a biomarker associated with a disease (e.g. cancer) to predict a disease or disease progression. The detection systems, devices and methods of the present invention are constructed to analyze a small amount of test sample and can be implemented by a user without extensive laboratory training.

Other expanded applications outside of the field of food safety include in-field use by military organizations, testing of antibiotics and biological drugs, environmental testing of products such as pesticides and fertilizers, testing of dietary supplements and various food components and additives prepared in bulk such as caffeine and nicotine, as well as testing of clinical samples such as saliva, skin and blood to determine if an individual has been exposed to significant levels of an individual allergen.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

A number of possible alternative features are introduced during the course of this description. It is to be understood that, according to the knowledge and judgment of persons skilled in the art, such alternative features may be substituted in various combinations to arrive at different embodiments of the present invention.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production: any method of use: etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1: Testing Filter Materials and Filtering Efficiency

Various filter materials and their combinations are tested for filtering efficiency and effects on signal measurement, for example, the loss of detection agents (SPNs). Commercially available filter materials such as membranes (PES, glass fiber, PET, PVDF, etc.), cotton, sand, mesh and silica are tested.

A filter including a combination of different filter materials is assembled. In one example, the filter assembly is composed of cotton and glass filter with a pore size of 1 μm. The cotton depth filter and paper filter are constructed to filter the sample sequentially. The filter assembly is tested for filtering different food matrices. The recovery of proteins and SPNs during the filtering process is measured. Various cotton volumes are used to construct the depth filters and the cotton depth filters are combined with membrane filters. These filter assemblies are tested for filtration efficiency and SPN recovery. In one study, 0.5 g of a food sample is collected and homogenized in 5 mL EPPS buffer (pH 8.4) (Tween 0.1%) and the homogenized food sample is incubated with 5 nM SPNs (signaling polynucleotides) labeled with Cy5 that is specific to an allergen protein. After incubation, a portion of the mixture is run through the filter assemblies and the recovery of proteins and SPNs is measured and compared with the pre-filtering measurements.

The filters are further tested and optimized to ensure efficiency of filtration and avoidance of significant SPN loss. In addition to testing different filter materials and their combinations, other parameters such as pore sizes, filtering areas (e.g., surface area/diameter, height of the depth filter), filtering volumes, filtration time and pressure required to drive the filtering process, etc., are also tested and optimized for various food matrices.

In one study, bleached cotton balls are used to assemble the depth filters with different filter volumes. Cotton filters with different ratios of width (i.e. diameter) and height are constructed; each model has a ratio of width and height ranging from about 1:30 to about 1:5. The cotton depth filters are then tested for filtration efficiency with different food masses and buffer volumes. In another study, these model cotton filters are assembled together with a PET membrane filter with 1 μm pore size and about 20 mm² filtrating area. Various food samples are homogenized and filtered through each filter assembly using different volumes of buffer. The filtrates are collected and the percentage of recovery is compared for each condition.

In another study, food samples are spiked with or without 50 ppm peanut. The spiked samples are homogenized, for example using the rotor 340 (e.g., as illustrated in FIGS. 3B and 3C) and the extractions are mixed with SPNs that specifically bind to peanut allergen. The SPN contains a Cy5 label at the 5′ end of the sequence. The mixture is filtered through a depth filter (e.g., a depth filter made of cotton) and a membrane filter (pore size: 1 μm). Fluorescence signals are measured and compared with the measurements of the pre-filtered mixture.

In separate studies, several parameters of each filter assembly are tested and measured including the pressure and time required for filtering, protein and nucleic acid binding, washing efficiency and assay compatibility and sensitivity. The assay compatibility is measured as the baseline intensity.

### Example 2: MgCl₂ Formulations

Several solid MgCl₂ formulations were tested to replace the addition of MgCl₂ solution after the sample homogenization in extraction buffer. The following characteristics of each formulation tested are evaluated: (1) the time to dissolve; (2) the final concentration of dissolved MgCl₂; (3) the effect of additives in the formulations on the detection assay; (4) no agitation required to dissolve; and (5) no breakup into powder and not blocking the outlet of the homogenization chamber.

### Lyophilized MgCl₂ Formulation

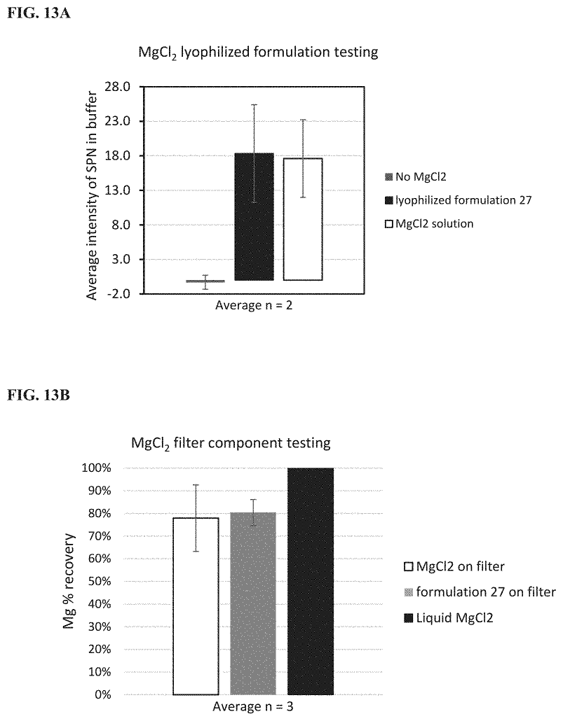

A total of 34 MgCl₂ formulations were lyophilized in 1.5 mL Eppendorf tubes and tested for dissolution time, mechanical stability, exposure to the extraction buffer for 10 seconds without agitation, and other features. Among these formulations are 2 formulations which rapidly dissolve and do not form powder. Several MgCl₂ formulations were exposed to the extraction buffer for 10 seconds without agitation and the magnesium content in the recovered buffer was determined by a BioVision Magnesium assay and the assay as described herein. The assay results indicate that the lyophilized MgCl₂ formulation comprising maltodextrin and hydroxyethylcellulose (HEC) (Table 1) gives the highest intensity signals of SPNs in buffer as shown in FIG. 13A.

### MgCl₂ as a Filter Component

MgCl₂ formulations (Table 1) were deposited on a cotton filter and dried at 60° C. The extraction buffer was pulled through the cotton filter with 1 psi vacuum. The percentage of magnesium recovered in filtrate was measured by the BioVision colorimetric magnesium assay. The MgCl₂ formulation comprising maltodextrin and hydroxyethylcellulose (HEC) (Table 1) was compared with what was recovered in MgCl₂ solution and MgCl₂ on the filter (FIG. 13B).

### MgCl₂ as Film

A total of 10 different MgCl₂ formulations were deposited on polystyrene supports and cured. The dissolution time was measured and all formulations dissolved in 10 seconds. The results indicate that none of the formulations have a strong adhesion to the polystyrene support.

TABLE 1
Components of MgCl₂ formulations
Formulations containing 1.0% glycerol

| | | | |
|---|---|---|---|
| glycerol | 1.0% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |

Formulations containing 0.7% glycerol

| | | | |
|---|---|---|---|
| glycerol | 0.7% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |

Formulations containing 0.5% glycerol

| | | | |
|---|---|---|---|
| glycerol | 0.5% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |
| PEG | 2.0% | glycine | 2.5% |
| PEG | 5.0% | glycine | 2.5% |
| maltodextrin | 0.5% | HEC | 0.1% |

Based on the test results, several fast-dissolving solid MgCl₂ formulations are selected (as shown in Table 2). The dissolution time for the filter deposition is dependent on flow rate. When the fastest flow rate was tested, the solid formulation dissolved in 10 seconds (as shown in Table 2).

TABLE 2
Fast-dissolving and mechanically robust solid MgCl₂ formulations

| | Lyophilized | | |
|---|---|---|---|
| | pellet | Film | Incurred in filter |
| Leading formulation | 0.5% glycerol/ | 1% maltodextrin/ | 1% maltodextrin/ |
| | 0.5% sucrose | 0.1% hydroxyethyl | 0.1% hydroxyethyl |
| | | cellulose | cellulose |
| Time for resuspension | 12 Seconds | 16 seconds | 10 seconds |
| Stability following | − | + | N/A |
| agitation (vortex 1 | | | |
| minute) | | | |
| Mg recovery in 10 | 100% | 100% | 80% |
| seconds (compared to | | | |
| MgCl₂ solution) | | | |

1. An assembly for detecting a molecule of interest in a sample, the assembly comprising:
(i) a sample processing cartridge configured to accept the sample for processing to a state permitting the molecule of interest to engage in an interaction with a detection agent comprising:
A sample receiving chamber with a homogenizer configured to homogenize the sample with an extraction buffer in the presence of the detection agent, thereby permitting the molecule of interest in the sample to engage in the interaction with the detection agent; and
a detection chamber with a window, wherein the detection chamber includes a separate substrate with a detection probe molecule immobilized thereon; and
(ii) a detector unit configured to accept the sample processing cartridge in a configuration which permits a detection mechanism housed by the detector unit to detect the interaction of the molecule of interest with the detection agent, wherein the interaction triggers a visual indication on the detector unit that the molecule of interest is detected.

2. The assembly of claim 1, wherein the molecule of interest is an allergen.

3. The assembly of claim 1, wherein the detection agent is an antibody or variant thereof, a nucleic acid molecule, or a small molecule.

4. (canceled)

5. The assembly of claim 3, wherein the detection agent is a signaling polynucleotide (SPN) derived from an aptamer that comprises a nucleic acid sequence that binds to the molecule of interest.

6. The assembly of claim 1, wherein the sample processing cartridge further comprises:
a first conduit to transfer the homogenized sample and detection agent through a filter system to provide a filtrate containing the molecule of interest and the detection agent; and
a second conduit to transfer the filtrate to a detection chamber with a window, wherein the detection mechanism of the detector unit analyzes the detection chamber through the window to identify the interaction of the molecule of interest with the detection agent in the detection chamber.

7. The assembly of claim 1, wherein the homogenizer is powered by a motor located in the detector unit, wherein the motor is functionally coupled to the homogenizer when the sample processing cartridge is accepted by the detector unit.

8. The assembly of claim 1, wherein the sample processing cartridge further comprises a chamber holding wash buffer for washing the detection chamber and a waste chamber for accepting and storing outflow contents of the detection chamber.

9. The assembly of claim 8 wherein the sample processing cartridge further comprises a rotary valve switching system configured to transfer the homogenized sample and detection agent through the filter system, to transfer the filtrate to the detection chamber, and to transfer the wash buffer to the detection chamber and outflow contents from the detection chamber to the waste chamber.

10. The assembly of claim 9, wherein the rotary valve switching system is further configured to provide a closed position to prevent fluid movement in the sample processing cartridge.

11. The assembly of claim 1, wherein the substrate is transparent and wherein the detection probe molecule immobilized thereon is configured to engage in a probe interaction with the detection agent, wherein the interaction of the molecule of interest with the detection agent prevents the detection agent from engaging in the probe interaction with the detection probe.

12. The assembly of claim 11, wherein the transparent substrate further comprises least one optically detectable control probe molecule immobilized thereon, for normalization of signal output measured by the detection mechanism.

13. (canceled)

14. The assembly of claim 12, wherein the detection agent includes an optically-detectable moiety which is activated when the probe interaction is engaged.

15. The assembly of claim 14, wherein the optically-detectable moiety is a fluorescent moiety.

16. The assembly of claim 15, wherein the detection mechanism housed by the detector unit is a fluorescence detection system with a laser for excitation of fluorescence that is configured to detect a fluorescence emission signal and/or a fluorescence scatter signal from the detection chamber.

17. (canceled)

18. The assembly of claim 16, wherein the detector unit further comprises a signal processor for analyzing the fluorescence emission signal and the fluorescence scatter signal to identify the probe interaction and transmit the identity of the molecule of interest, to the visual indication such that an operator of the assembly is informed of the presence or absence of the molecule of interest in the sample.

19. (canceled)

20. The assembly of claim 1, wherein the sample processing cartridge further comprises a sample concentrator for concentrating the filtrate prior to transfer of the filtrate to the detection chamber.

21. The assembly of claim 1, further comprising a sampler, the sampler comprising a hollow tube with a cutting edge for cutting a source to generate and retain the sample within the hollow tube and a plunger for pushing the sample out of the hollow tube and into a port in the sample processing cartridge.

22.-26. (canceled)

27. The assembly of claim 11, wherein the detection probe is a nucleic acid molecule comprising a nucleic acid sequence that is complementary to the nucleic acid sequence of the detection agent.

28.-29. (canceled)

30. The assembly of claim 27, wherein the detection probe is immobilized in a local area of the substrate that is referred to a reaction area and wherein the control probe is immobilized in a separate local area of the substrate that is referred to as a control panel.

31. (canceled)

32. The assembly of claim 16, wherein the fluorescence detection system comprises (i) a laser for excitation of fluorescence; (ii) a plurality of optical elements to guide the laser excitation to the substrate within the detection chamber; (iii) a plurality of collection lens to collect the fluorescence emitted from the substrate; (iv) a fluorescence detector for measuring the emitted light from the substrate; and (v) a signal processor for analyzing fluorescence emission signal and/or fluorescence scatter signal to identify the probe interaction and transmit the identity of the allergen of interest to the visual indication such that an operator is informed of the presence or absence of the allergen of interest in the sample.

33. The assembly of claim 32, wherein the optical elements of the fluorescence detection system are placed within a stepped bore in the detector unit in either a straight or a folded arrangement.

34. The assembly of claim 10, wherein the rotary valve motor comprises a DC gear motor with two optical sensors: an output optical sensor and a direct shaft optical sensor; and a microcontroller comprising an output coupling and encoder wheel, a direct motor shaft and a direct shaft encoder wheel.

35. A sample processing cartridge for processing a sample for detection of a molecule of interest in the sample comprising:
(i) a sample receiving chamber with a homogenizer configured to homogenize the sample with an extraction buffer in the presence of a detection agent, thereby permitting the protein of the interest in the sample to engage in the interaction with the detection agent,
(ii) a filter system configured to provide a filtrate containing the molecule of interest and the detection agent,
(iii) a detection chamber with a window, wherein the detection chamber includes a separate substrate with a detection probe molecule immobilized thereon,
(iv) a chamber holding wash buffer for washing the detection chamber,
(v) a waste chamber for accepting and storing outflow contents of the detection chamber,
(vi) a rotary valve switching system and conduits configured to transfer the homogenized sample and detection agent through the filter system, to transfer the filtrate to the detection chamber, and to transfer the wash buffer to the detection chamber and outflow contents from the detection chamber to the waste chamber, and
(vii) an air flow system configured to regulate air pressure and flow rate in the cartridge.

36. The sample processing cartridge of claim 35, wherein the detection agent is a nucleic acid molecule comprising a nucleic acid sequence that binds to the allergen of interest, and a fluorescent moiety.

37. (canceled)

38. The sample processing cartridge of claim 36, wherein the detection probe molecule immobilized on the substrate is configured to engage in the interaction with the detection agent, wherein the interaction of the allergen of interest with the detection agent prevents the detection agent from engaging in the probe interaction with the detection probe.

39. The sample processing cartridge of claim 38, wherein the detection probe is a nucleic acid molecule comprising a nucleic acid sequence that is complementary to the nucleic acid sequence of the detection agent.

40. The sample processing cartridge of claim 39, wherein the substrate further comprises at least one optically detectable control probe molecule immobilized thereon, for normalization of signal output measured by the detection mechanism.

41. (canceled)

42. The sample processing cartridge of claim 35, wherein the substrate is a glass chip, or a plastic chip, or a membrane-like chip.

43. The sample processing cartridge of claim 35, wherein the filter system is composed of a bulk filter comprising a cotton volume and a PET membrane filter.

44. (canceled)

45. The sample processing cartridge of claim 43, wherein the PET membrane has a pore size of 1 μm.

46. The sample processing cartridge of claim 35, wherein the rotary valve switching system is further configured to provide a closed position to prevent fluid movement in the cartridge.

47. (canceled)

48. The sample processing cartridge of claim 35, wherein the cartridge is made of polymers having minimal auto-fluorescence.

49.-67. (canceled)

68. A system for detecting the presence or absence of an allergen in a sample, the system comprising:
a device comprising an optical system configured to measure fluorescence signal outputs, thereby detecting the presence or absence of the allergen; and
a disposable cartridge configured to process the sample, which docks into a receptacle of the device, the cartridge comprising:
(i) an upper module comprising a plurality of chambers isolated from each other with each chamber of the plurality of chambers comprising a lower port to permit entry and/or exit of fluids, the plurality of chambers comprising:
(1) a homogenization chamber including a homogenizer for extracting the allergen from the sample with in an extraction buffer;
(2) a wash buffer chamber;
(3) a waste chamber configured to receive liquid waste; and
(4) a detection chamber in optical communication with the optical system, for detecting the allergen; and
(ii) a base module configured to connect to the upper module, the base comprising:
(1) a plurality of fluid paths joining the lower port of each chamber when the cartridge is inserted into the receptacle; and
(2) a valve configured to form a plurality of bridging fluid connections between individual fluid paths of the plurality of fluid paths, thereby allowing selective fluid movement into and/or out of the plurality of chambers.

69. The system of claim 68, wherein the valve is a rotary valve driven by a motor located in the device, the motor comprising one or more optical sensors for determining positions of the rotary valve.

70. The system of claim 69, wherein the plurality of bridging fluid connections comprises:
(a) a first fluid connection between the wash buffer chamber and the reaction chamber; and
(b) a second fluid connection between the homogenization chamber and the detection chamber.

71. The system of claim 70, wherein the cartridge further comprises:
(iii) a filter assembly and a filter fluid path between the homogenization chamber and the filter assembly to obtain a filtered sample after the sample is homogenized in the homogenization chamber; and
(iv) a filtrate chamber for holding the filtered sample.

72. The system of claim 71, wherein the second fluid connection includes the filtrate chamber between the homogenization chamber and the detection chamber and wherein the rotary valve is configured to make the second fluid connection between the filtrate chamber and the detection chamber.

73. The system of claim 72, wherein the rotary valve includes a position where all bridging fluid connections are closed.

74. The system of claim 73, wherein the upper module further comprises an extraction buffer reservoir and a fluid channel extending from the extraction buffer reservoir to the homogenization chamber.

75. The system of claim 74, wherein the detection chamber includes a substrate containing a detection probe molecule immobilized thereon; the substrate configured to detect the allergen.

76. The system of claim 75, wherein the substrate is a glass chip with a nucleic acid detection probe anchored thereto, the nucleic acid probe hybridizing to a free signaling polynucleotide (SPN) having a fluorescent probe attached thereto, the SPN comprising a nucleic acid sequence that specifically binds to the allergen, wherein, when bound to the allergen, the SPN does not bind to the nucleic acid probe.

77. The system of claim 76, wherein the glass chip comprises at least two control panels printed with oligonucleotide sequences that do not bind to the SPN or the allergen of interest in the sample.

78.-80. (canceled)
